(19) United States
(12) Patent Application Publication
(10) Pub. No.: US 2014/0093953 A1
(43) Pub. Date: Apr. 3, 2014

(54) NON-ADHERENT CELL SUPPORT AND MANUFACTURING METHOD

(71) Applicant: The Regents of the University of Michigan, Ann Arbor, MI (US)

(72) Inventors: Patrick Neal Ingram, Ann Arbor, MI (US); Euisik Yoon, Superior Township, MI (US)

(73) Assignee: The Regents of the University of Michigan, Ann Arbor, MI (US)

(21) Appl. No.: 14/043,442

(22) Filed: Oct. 1, 2013

Related U.S. Application Data
(60) Provisional application No. 61/708,625, filed on Oct. 1, 2012.

(51) Int. Cl. G01N 33/50

(52) U.S. Cl. CPC G01N 33/5005
USPC 435/299.1; 435/289.1; 156/272.6; 216/67

(57) ABSTRACT

A non-adherent cell support for use as a substrate in fluidic chambers used for cell culturing and assays. The non-adherent cell support allows for the formation of sphere cultures from single cells, which can better mimic primary tumor-like behavior in the study of cancer stem cells. The non-adherent cell support can allow for adhesive culturing and may include a hydrophobic substrate having a lower body and a raised support structure extending upwardly from an upper surface of the body. The support structure comprises one or more vertically extending support members that extend from a proximal portion at the upper surface of the body to a distal end spaced from the upper surface of the body. The support structure may be formed from a biocompatible material such as poly-2-hydroxyethyl methacrylate, polydimethylsiloxane, polymethyl methacrylate, polystyrene, or a polyethylene glycol diacrylate-based hydrogel.

### CROSS-REFERENCE TO RELATED APPLICATION

This application claims the benefit of U.S. Provisional Application Ser. No. 61/708,625 filed on Oct. 1, 2012, the entire contents of which are hereby incorporated by reference.

### TECHNICAL FIELD

This invention relates generally to substrates and fluidic chambers used for cell culturing and assays.

### BACKGROUND

Cell culture is the process by which cells are grown under controlled conditions, generally outside of their natural environment. In practice, the term “cell culture” has come to refer to the culturing of cells derived from multi-cellular eukaryotes, especially animal cells. Cells can be grown either in suspension or adherent cultures. Adherent cells require a surface, such as a tissue culture plastic, which may be coated with extracellular matrix components to increase adhesion properties and provide other signals needed for growth and differentiation. Most mammalian cells derived from solid tissues are adherent in nature. However, there are many applications where non-adherent mammalian culture is desirable, such as with embryonic stem cells, neural stem cells, and macrophages. In these situations, cells can be grown as non-adherent cell clusters, known as spheroids. Applications for these spheroids include cancer drug screening tools.

Cell heterogeneity is a hallmark of multi-cellular life with heterogeneity being provided by asymmetric and symmetric division, and cancer has been shown to be no different. While heterogeneity may manifest in many ways, the presence and behavior of cancer subtypes known as cancer stem cells (CSCs) or tumor initiating cells (TICs) are of great interest when screening cancer targeting therapeutic agents. These CSCs/TICs are linked to drug resistance in cancer and may be the culprit for reemergence after therapy. Drugs that target and selectively remove these drug resistance sub-populations have been shown to have great therapeutic potential.

However in many cancers, there is considerable evidence that several subpopulations of CSCs/TICs may exist within one tumor, and that their identification would require the use of many cell markers in combination with other identifying characteristics. Accordingly, there is a need for easier methods for enriching and studying this behavior for drug screening applications, as these markers can be different even among seemingly similar cell types. Additionally, 2D culture screening methods currently used do not correlate well with clinical responses due to morphology and gene expression differences. Current high throughput heterogeneous screening methods are unable to easily identify CSCs/TICs in many cancer types or place them in an environment that can provide clinically relevant results.

Non-adherent, or suspended, sphere culture of cancer cells has been shown to better mimic primary tumor-like behavior. In addition, suspension sphere cultures can be used to enrich CSCs and characterize CSCs from multiple cell types. Non-adherent surfaces can selectively allow growth from CSCs through sphere formation, as a non-progenitor bulk tumor should not survive suspension environments. These 3D spheroid results provide for stronger correlations between drug effects and eventual patient outcomes.

Traditionally, biologically inert, low-cell binding dishes and plates are used for non-adherent culture. Often these plates utilize polymers with coatings presenting phosphorylcholine moieties that mimic the cell membrane surface, resulting in cultures that can be stable for well over 2 months. These modifications, however, are not compatible with microfabrication techniques. State-of-the art hanging drop spheroid culture methods are unable to facilitate the growth of spheres from single cells, and other methods that utilize non-adherent chemical coatings such as pluronics degrade over time and can disrupt natural sphere formation. Existing methods are either incapable of producing cultures from single cells or are inefficient and expensive. For example, suspension culture dishes are not compatible with microfabrication techniques. Furthermore, state-of-the art hanging drop spheroid culture methods are unable to grow spheres from single cells, instead needing as many as 20 to 100 to start growth. Methods that utilize non-adherent chemical coatings degrade in a matter of days and often inhibit natural sphere formation through the addition of hydrophobic molecules. Also, topographically patterned hydrophobic surfaces have recently been studied and have become popular for anti-biofouling applications (preventing bacterial and protein adhesion).

Microfabrication of microfluidic devices for cell assaying is generally known, with one example being disclosed in WO 2011/056643 which uses a glass substrate for the cell support within the fabricated microchambers.

### SUMMARY

According to one embodiment, there is provided a hydrophobic substrate having a lower body and a raised support structure extending upwardly from an upper surface of the body. The support structure comprises one or more vertically extending support members that extend from a proximal portion at the upper surface of the body to a distal end spaced from the upper surface of the body. The distal end of the one or more support members forms an interrupted support surface for hydrophobic support of cells on the support structure.

According to another embodiment, there is provided a substrate having a lower body and a hydrophobic support structure extending upwardly from an upper surface of the body. The support structure is formed from poly-2-hydroxyethyl methacrylate and comprises one or more support members that extend from a proximal portion at the upper surface of the body to a distal end spaced from the upper surface of the body.

According to another embodiment, there is provided a method of making a microfluidic device having a non-adherent cell support for use in cell assays, comprising the steps of fabricating one or more microfluidic chamber structures and a non-adherent cell support and joining one or more microfluidic chambers to the non-adherent cell support.

According to another embodiment, there is provided a method of making a microfluidic device having a non-adherent cell support for use in cell assays, comprising the steps of providing a silicon wafer, spin coating and patterning said silicon wafer with photoresist, deep reactive ion etching the coated silicon wafer to produce a patterned mold, pouring an uncured biocompatible material onto the patterned mold resulting in an uncured non-adherent cell support, curing the uncured non-adherent cell support, releasing the cured non-adherent cell support from the patterned mold, and joining the non-adherent cell support to one or more microfluidic chambers.

### BRIEF DESCRIPTION OF THE DRAWINGS

Preferred exemplary embodiments will hereinafter be described in conjunction with the appended drawings, wherein like designations denote like elements, and wherein:

FIG. 1 is a three-dimensional partially transparent perspective view of an embodiment of a microchamber constructed in accordance with the present invention;

FIG. 2 is an elevational view of the microchamber of FIG. 1 showing its front valve and tapered opening;

FIG. 3 depicts a sequential cell loading process for introducing individual cells into the microchamber of FIG. 1;

FIG. 4 is a schematic diagram showing an array chip of microchambers along with a perspective diagrammatic view of a single microchamber which can be fabricated using a non-adherent cell support as described herein;

FIG. 5 is an image of a fabricated microchamber array such as is shown in FIG. 4;

FIG. 6 is a schematic diagram in accordance with another embodiment showing an array chip of microchambers along with a perspective diagrammatic view of a single microchamber which can be fabricated using a non-adherent cell support as described herein;

FIG. 7 is a confocal laser microscopy image of a fabricated non-adherent cell support with a honeycomb shaped pattern;

FIG. 7A is a cross-sectional perspective view of the confocal laser microscopy image of a fabricated non-adherent cell support taken along line A-A of FIG. 7;

FIG. 8 is a confocal laser microscopy image of a fabricated non-adherent cell support with a hollow pillar shaped pattern;

FIG. 9 depicts a procedure for fabricating the non-adherent cell support in accordance with one embodiment;

FIG. 10 is a scanning electron microscope image of a mold that can be used to fabricate the non-adherent cell support;

FIG. 11 depicts a procedure for fabricating the non-adherent cell support in accordance with another embodiment;

FIG. 12 depicts a procedure for fabricating the non-adherent cell support in accordance with another embodiment;

FIG. 13 is an image of the non-adherent cell support formed by the procedure depicted in FIG. 12;

FIG. 14 is a bar graph showing the effects of a variety of cleaning methods on the surface contact angle of a non-adherent cell support;

FIG. 15 shows optical images of 10 μL droplets on hydrophobic surfaces with droplet contact angles and the corresponding patterns of molds used to fabricate the non-adherent cell support;

FIG. 16 diagrams the transition from a Cassie-Baxter state to a Wenzel state on a hydrophobic surface due to the body forces of the fluid overcoming the contact line forces;

FIG. 17 is a chart showing the variability of hydrophobicity of the non-adherent cell support, as measured by contact angle, depending on the size and pitch of cell support members;

FIG. 18 illustrates the steric expansion of poly-2-hydroxyethyl methacrylate (polyHEMA) polymer chains when exposed to water;

FIG. 19 depicts a polyHEMA barrier capable of preventing GFP-expressing MDA-MB-231 cells from migrating across the barrier;

FIG. 20 is a laser interferometer microscopy image of microchambers with a non-adherent cell support in accordance with one embodiment;

FIG. 21 is a laser interferometer microscopy image of a large scale array of the microchambers depicted in FIG. 20;

FIG. 22 shows depicts SUM159 cell growth in microchambers constructed in accordance with one embodiment of the disclosed method;

FIG. 23 shows C2C12 myoblast cultures grown on a non-adherent cell support with pillar shaped support members on the left and with a honeycomb shaped support member on the right;

FIG. 24 shows 10T1/2 fibroblast cultures grown on a non-adherent cell support with pillar shaped support members on the left and with a honeycomb shaped support member on the right;

FIG. 25 shows 6 days of growth of a SUM159 sphere culture on a non-adherent cell support;

FIG. 26 depicts captured SUM159 cells in a microchamber;

FIG. 27 shows a single cell derived spheroid formation in a microchamber;

FIG. 28 depicts captured skov3 cells and SUM159 cells in an array of microchambers;

FIG. 29 shows a captured skov3 cell in a single microchamber;

FIG. 30 depicts skov3 cells in adherent and suspension cultures;

FIG. 31 is a chart showing the cell viability of a single cell anoikis assay of skov3 cells for 6 days in polyHEMA treated microchambers for suspension culture;

FIG. 32 shows the development of a SUM159 sphere from a single cell in suspension culture inside a polyHEMA surface-coated microchamber over 10 days;

FIG. 33 is a graph showing sphere formation rates of sub-populations of T47D breast cancer cells;

FIG. 34 is a graph comparing semi-adherent and suspension sphere and colony formation of CD44+/CD24−, CD44−/CD24+, and CD44+/CD24+ cells; and

FIG. 35 is a graph depicting how single cell derived sphere formation may be used as a readout indicator for CSC-targeted drug screening.

### DETAILED DESCRIPTION OF THE ILLUSTRATED EMBODIMENTS

The non-adherent cell support disclosed herein allows for non-adherent cell culturing and assays using a hydrophobic support surface for the cell(s). Useful applications include single cell spheroid formation inside a high-throughput microfluidic chip capable of long term chemical free non-adherent mammalian cell culture. In certain applications, the non-adherent cell support may also allow for the adhesion of cells that require adhesion for successful culturing. Furthermore, the disclosed cell support and integrated microfluidic device presented here can be used to provide a low cost, high throughput, and novel approach for oncologists and other researchers to isolate and characterize rare CSC/TIC populations. The non-adherent cell support can also be used in both macro-scale chambers or in integrated microfluidic microchambers. Accordingly, as used herein, the term “chambers” includes macro-scale chambers, microchambers, wells or any other open or closed cell retention spaces used to culture or otherwise assay cells. Because the integration of the non-adherent cell support with one or more microfluidic microchambers can result in a high-throughput, the discussion below is primarily focused on its application in that context.

An integrated microfluidic platform automates single cell placement and permits easy tracking of single cells because the cells are geometrically confined in each microchamber inside the microfluidic device. In traditional culture plates, tracking single cells within the large area is very time consuming, extremely slow, and laborious. Furthermore, microchambers allow for continuous perfusion of culture media to the growing sphere. In the standard 96-well plate technique, media may only be changed by exposing the culture environment and replacing the media that has been lost through evaporation. This process causes imbalances in pH and solute concentrations, both of which are critical parameters for successful sphere formation. Within the one or more microchambers, it is possible to have a continuous perfusion of media through the microchambers by a gravity driven flow, constantly supplying fresh, well controlled nutrients in a manner that cannot be easily implemented using other traditional culture techniques.

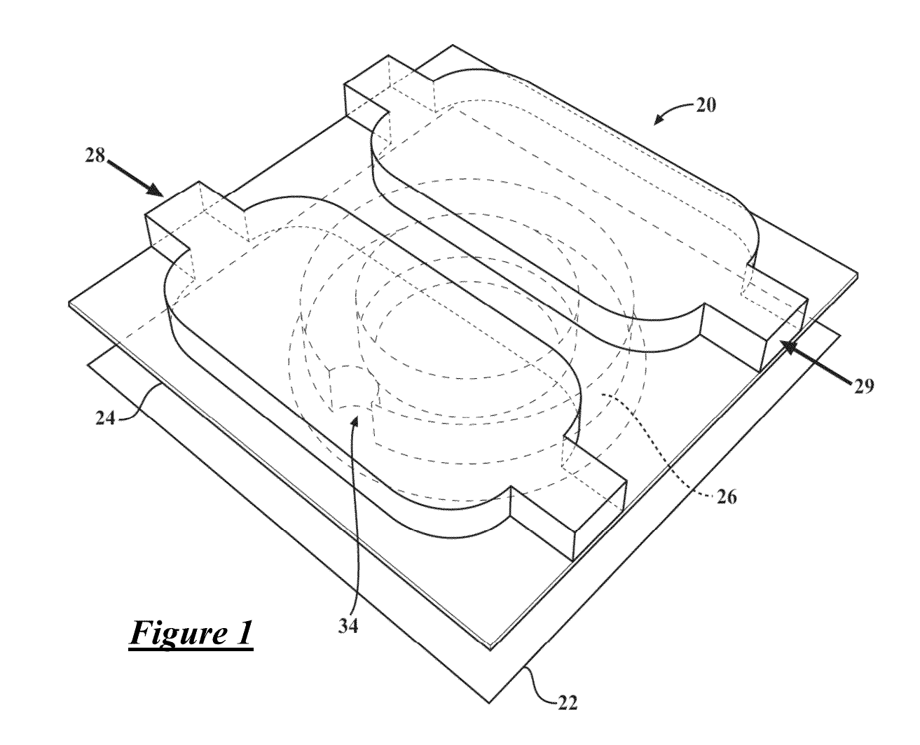

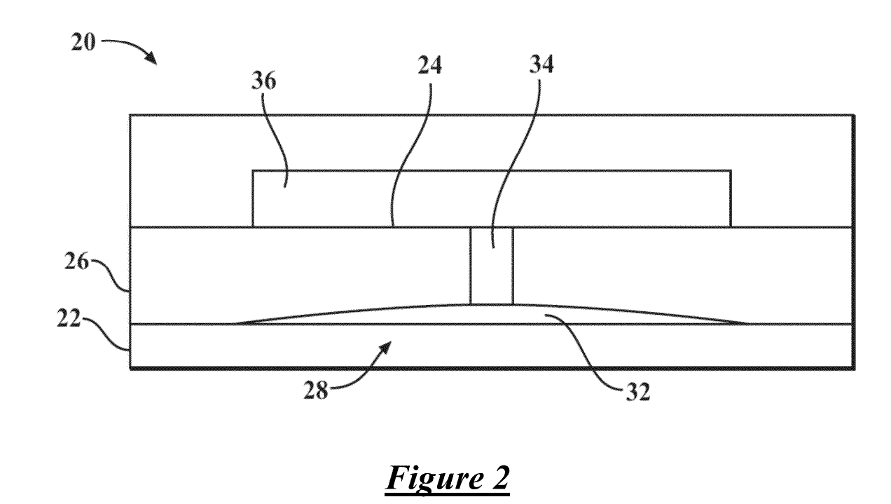

FIGS. 1-3 depict a microfluidic device having a single microchamber 20 that generally comprises a non-adherent cell support 22, chamber upper wall 24, a chamber sidewalls structure 26 that extends downwardly from the upper wall 24 towards the non-adherent cell support 22, and front and rear valves 28, 29 that control the injection and extraction of cells and perfusion of media into and out of the interior region 30 of the microchamber 20. In FIGS. 1-3, the chamber sidewall structure 26 comprises a single annular sidewall, although polygonal and other shaped sidewall structures can be used. As indicated in the elevation view of the front valve in FIG. 2, the sidewall structure 26 extends from the upper wall 24 to the non-adherent cell support 22 except at the valve 28 where it forms a tapered opening 32 that has a gap of about 5 μm at the maximum height of the opening 32 at the center of the valve. This gap varies from the maximum spacing at the center valve 28 down to zero at opposite ends of the valve where the sidewall meets the non-adherent cell support 22. As will be discussed below, operation of the valve 28 permits this gap to be increased, so as to admit cell(s) into the interior 30 of the microchamber 20, or decreased down to zero to thereby seal the microchamber 20 and provide complete environmental isolation of the cell(s) within the chamber. Although the front valve 28 is shown in FIG. 2, the rear valve 29 can have the same construction so as to include its own opening 33 through which cell(s) can be released from the microchamber 20. This initial gap in the openings 32, 33 of the valves permits the flow of media through the microchamber while preventing cell transference either into or out of the interior region 30 of the chamber. A cell capture site 34 is provided at the front valve 28 upstream of the media flow so that by inserting cell(s) into the flow stream, the cell capture site 34 can trap an individual cell while the fluid is flowing into the opening 32. Then, by activating the front valve 28 to fully open it, the cell can then be drawn by the flow stream into the interior region 30 of the chamber. The valve 28 can then be returned to its initial state (partially opened) to permit perfusion or can be closed completely along with the rear valve 29 to isolate the cell.

Thus, each valve provides a tri-state operation that includes a closed position, neutral (partially opened) position, and open position, with the neutral position for each valve permitting fluid flow through the valve while preventing cell transference through the valve, the open position for each valve permitting fluid flow and cell transference through the valve. Preferably, the microchamber 20 is made from a flexible material such that each valve 28, 29 can be pneumatically controlled via an actuator in the form of a respective fluid chamber 36, 37 positioned above the section of sidewall 26 located at its associated tapered opening 23, 33; see, for example, the front valve 28 as shown in FIG. 2. The microchamber 20 is constructed such that the valves 28, 29 are in their neutral position when the microchamber is in a relaxed state; that is, when each valve's activating fluid chamber is neither pressurized nor partially evacuated. Then, by partially evacuating the fluid chamber, the chamber sidewall is drawn upwardly thereby increasing the cap at the valve opening to a size sufficient to admit a cell into the microchamber. Or, by applying a positive pressure to the fluid chamber, the chamber sidewall is forced downward into sealing engagement with the non-adherent cell support 22.

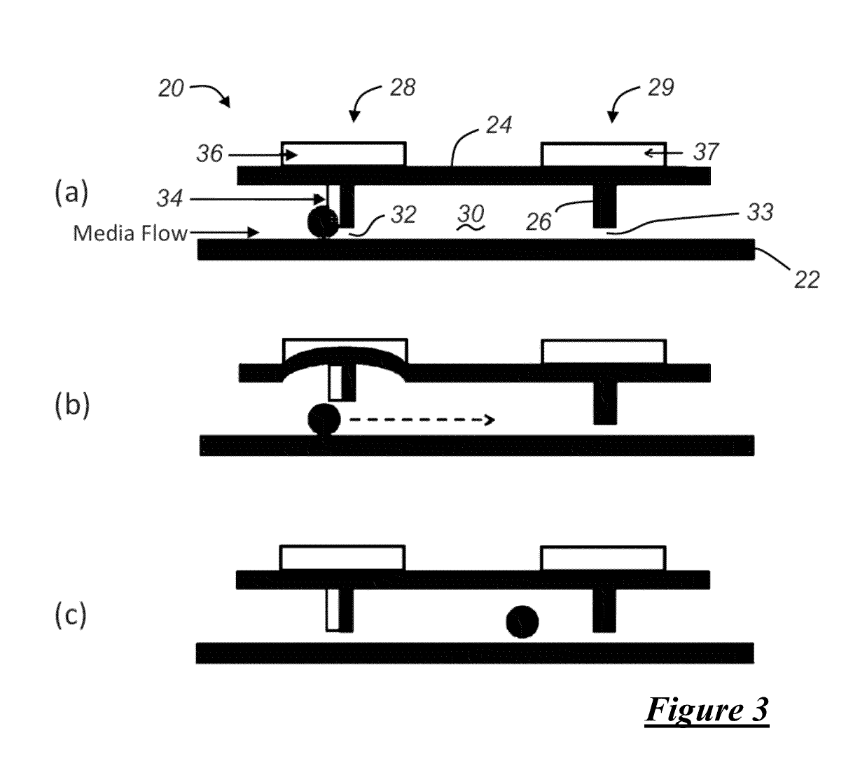

This operation of the valves to sequentially capture two individual cells is shown in FIG. 3. The first step is to flow fluid containing injected cells across the non-adherent cell support 22 while maintaining the valves 28, 29 in their neutral position. The results in the fluid flowing through the microchamber 20 such that an injected cell is trapped at the capturing site 34 during the flow. This is shown at (a) in FIG. 3. Then, at (b), the front valve 28 is actuated to its open position which permits the trapped cell to move into the interior region 30 of the chamber under the drag force of the fluid flow. This is done while maintaining the rear valve 29 in its neutral position. Then, at (c), the front valve 29 is returned to its neutral position. Again, this valve activation is carried pneumatically using the pneumatic (or air) chamber 36 shown in FIG. 3. The non-adherent cell support 22 can act as a hydrophobic substrate allowing for successful single cell sphere cultures without adhering to the bottom of the chamber. Additional information concerning the structure and use of the portions of microchamber 20 other than the cell support 22 can be found in WO 2011/056643, the disclosure of which is hereby incorporated by reference.

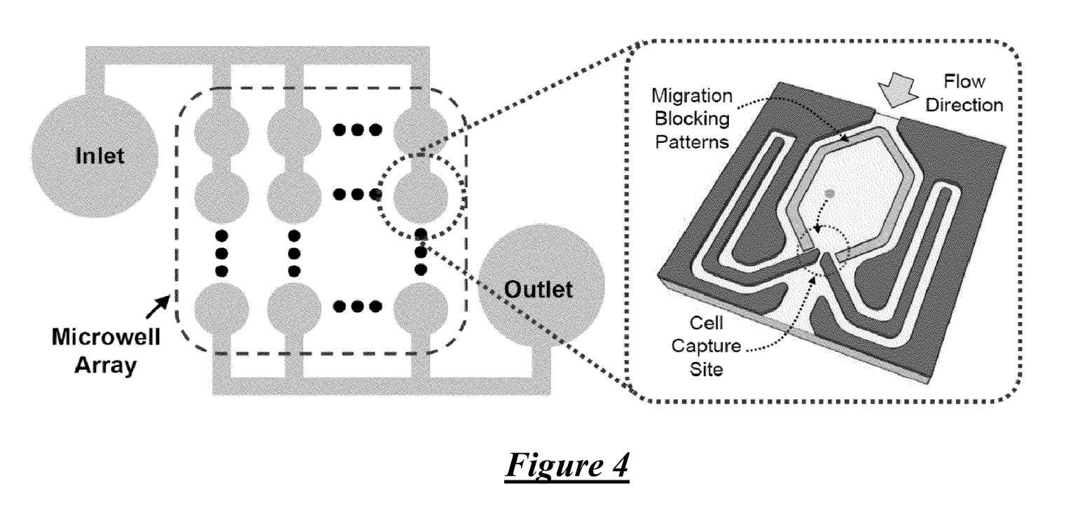

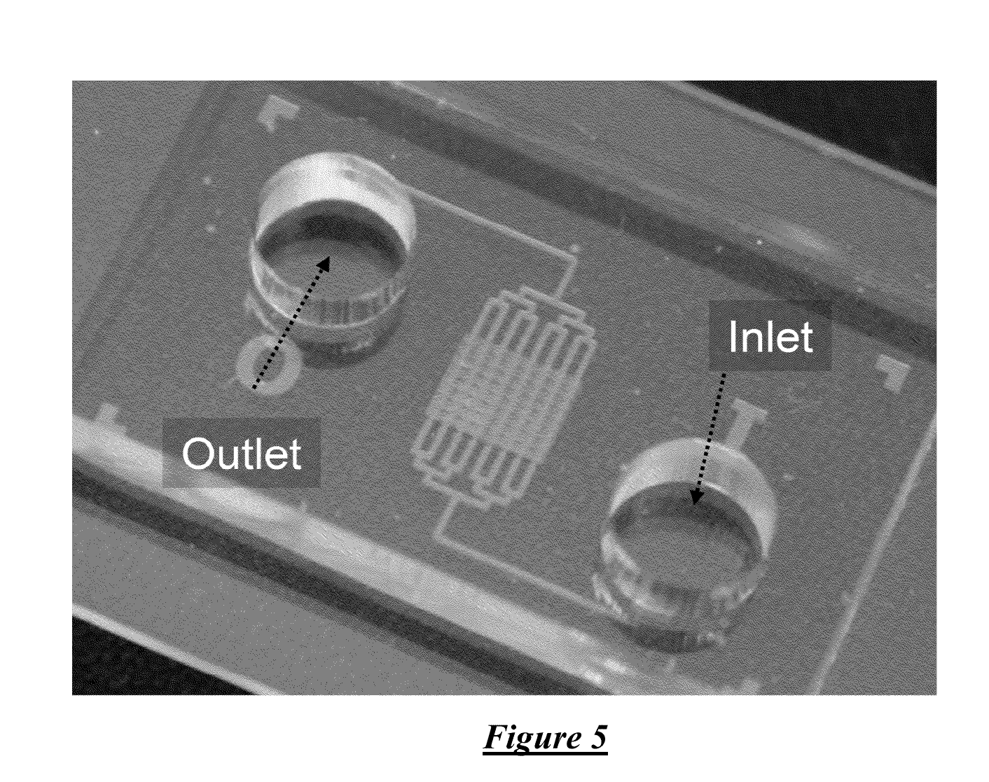

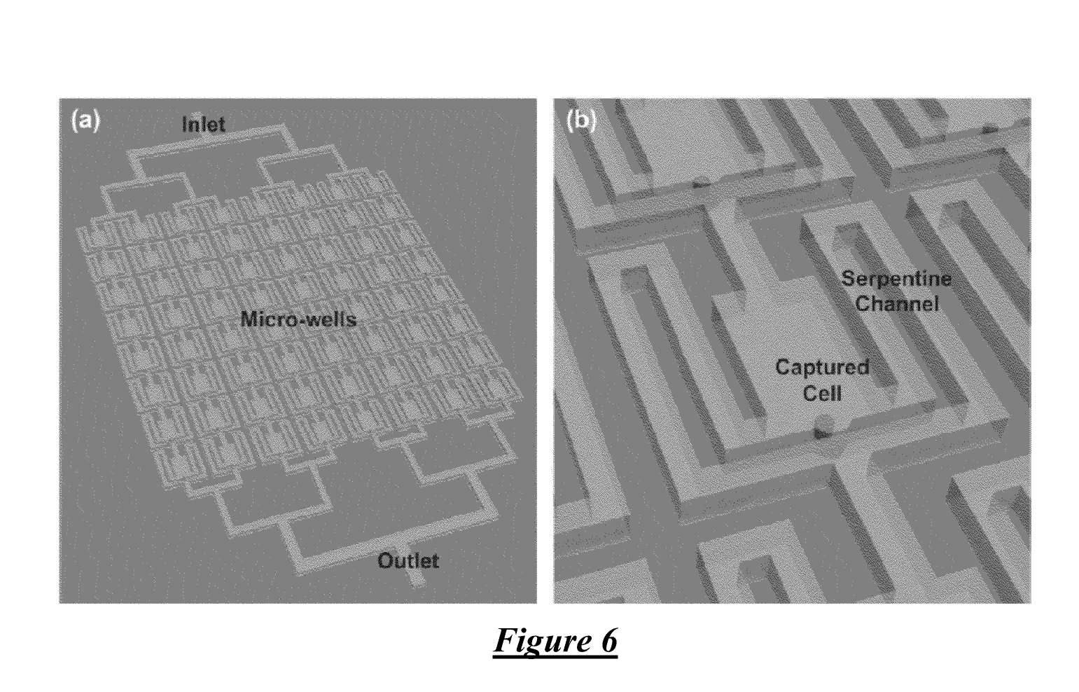

FIG. 4 depicts another embodiment that comprises a chip array of microchambers utilizing a hydrophobic substrate such as cell support 22, which is described in further detail below. The array chip has a hydrodynamic guiding structure in each unit microwell (microchamber) to increase capturing efficiency. By using a simple gravity flow (from uneven media level between the inlet and outlet reservoirs), the device can maintain a continuous flow for the entire duration of the experiments. This makes cell loading and operation simple without the need of any external controls. To prohibit possible cell migration between adjacent microwells, pluronic copolymer (F108) may be coated on the walls along the microwell boundaries to block cell adhesion. FIG. 5 shows a fabricated 8×8 microwell array (40 μm in channel height and 200 μm×200 μm in microwell size) according to the general design depicted in FIG. 4. As in the embodiment of FIGS. 1-3 above, a hydrophobic cell support having an interrupted, non-adherent surface is used rather than a glass plate as has been used in prior art devices. Similarly, FIG. 6 depicts another embodiment that comprises a chip array of microchambers utilizing a hydrophobic substrate such as cell support 22 described in more detail below. A single microchamber is shown enlarged on the right. In this particular embodiment, the cell support consists of poly-2-hydroxyethyl methacrylate (polyHEMA) formed on a glass plate.

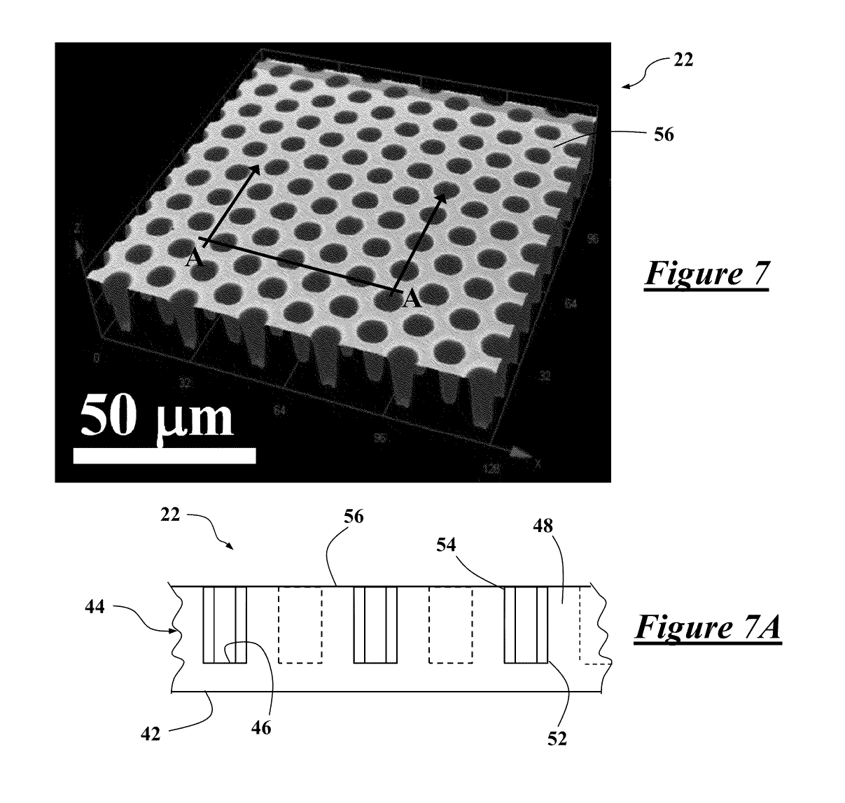

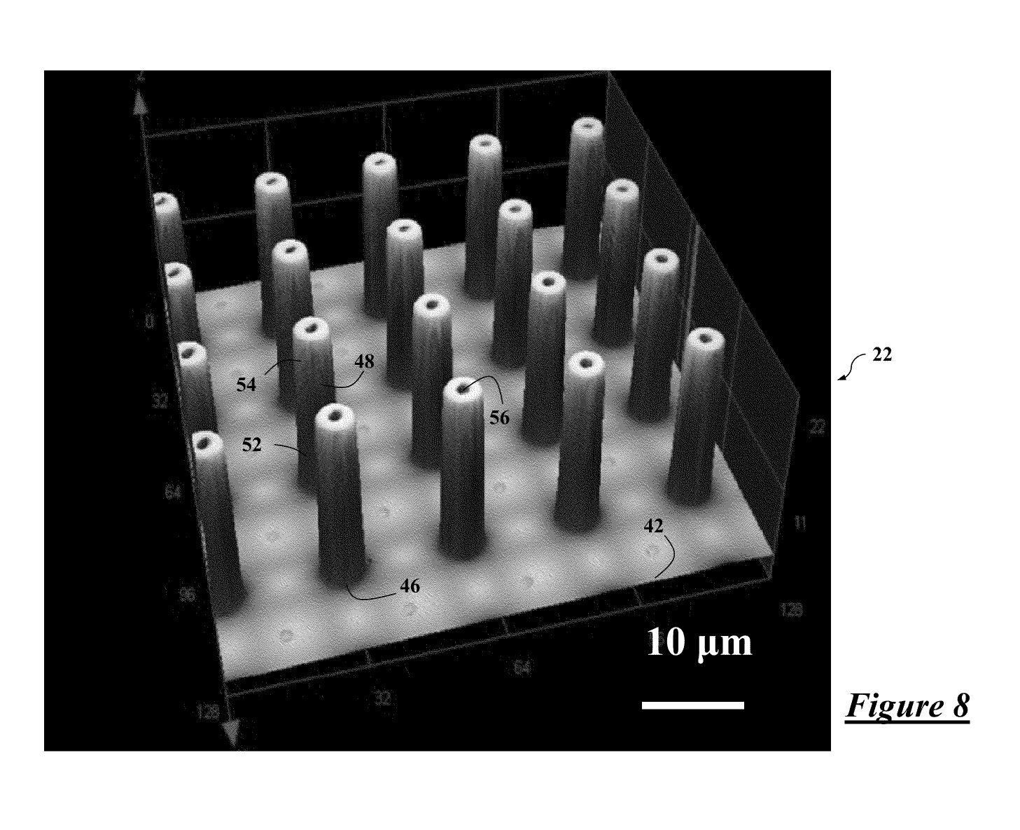

FIG. 7 shows a confocal laser microscopy image of one embodiment of a non-adherent cell support 22. FIG. 7A shows a partial cross-section of this embodiment taken along line A-A of FIG. 7. The upper hydrophobic surface is depicted with the remaining solid body being transparent, and the hexagonal wells shown extending down into the solid body. FIG. 8 shows a confocal laser microscopy image of one embodiment of a non-adherent cell support 22 with hollow pillar shaped vertically extending support members 48. As shown in FIG. 8 and in the partial cross-sectional perspective of FIG. 7A, the non-adherent cell support 22 comprises a hydrophobic substrate having a lower body 42 and a raised support structure 44 extending upwardly from an upper surface 46 of the lower body 42. The raised support structure 44 is comprised of one or more vertically extending support members 48 that extend from a proximal portion 52 at the upper surface 46 of the lower body 42 to a distal end 54 spaced from the upper surface 46 of the lower body 42. The distal end 54 of the one or more support members 48 form an interrupted support surface 56 for hydrophobic support of cells on the support structure 22.

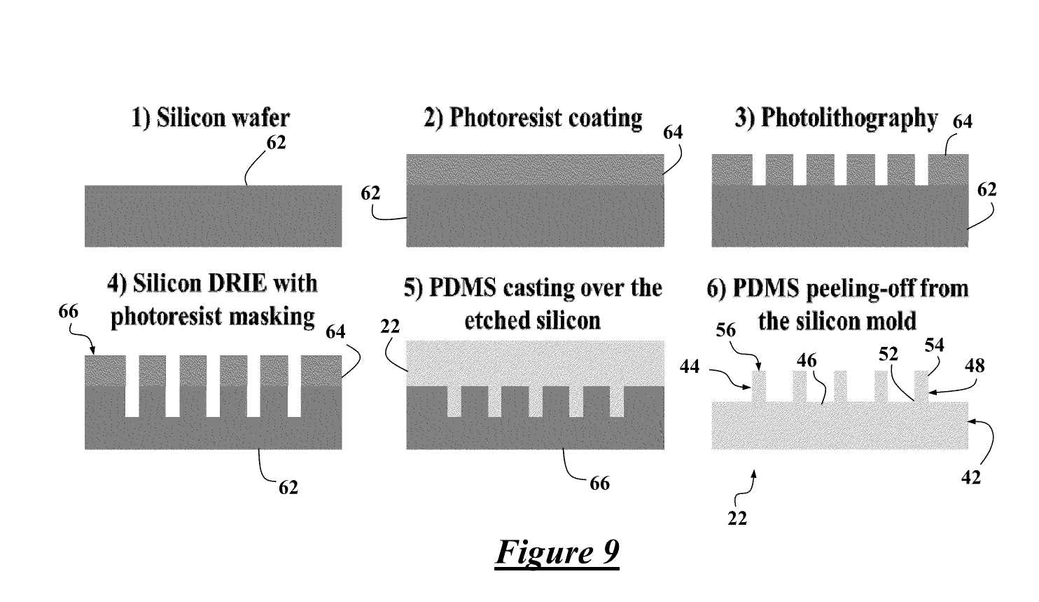

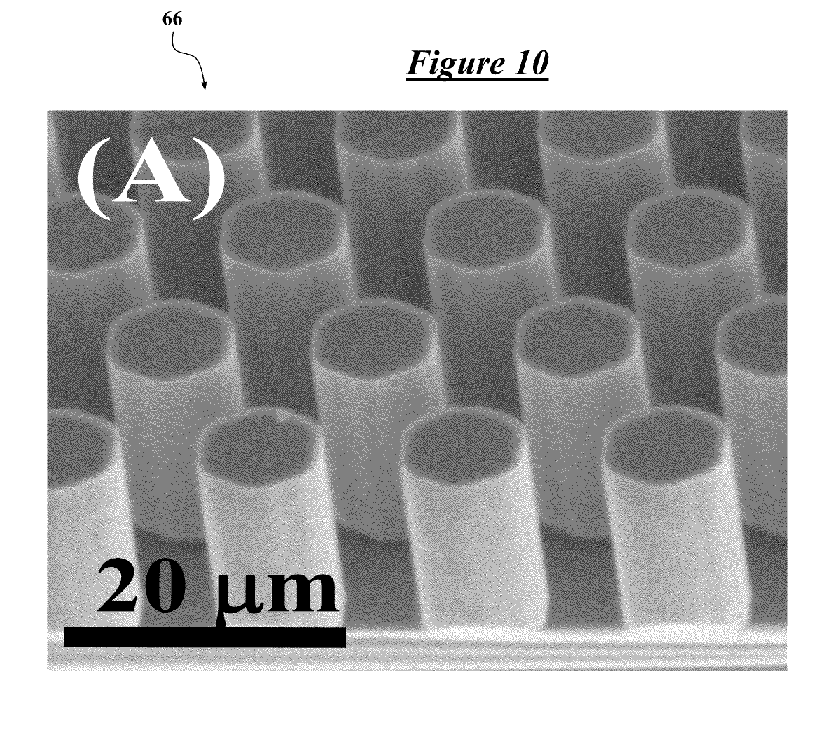

One potential way of making a non-adherent cell support 22 is shown in FIG. 9 and FIG. 10. FIG. 9 depicts one embodiment of a soft lithography method of forming the non-adherent cell support 22 with a biocompatible material. The cross-sections of FIG. 9 are diagrammatic only and not intended to represent the specific honeycomb structure of FIG. 7 or the pillar structure of FIG. 8. This particular embodiment uses polydimethylsiloxane (PDMS) as the biocompatible material. However, as will be apparent to one having ordinary skill in the art, any biocompatible material suitable for use as a non-adherent cell support can be used, such as polyHEMA, polymethyl methacrylate (PMMA), polystyrene (PS), or a polyethylene glycol diacrylate-based hydrogel (PEGDA). As shown in steps (1)-(3) of FIG. 9, a silicon wafer 62 is provided and then subjected to spin coating and patterning with photoresist 64. The silicon wafer with the photoresist masking is then subjected to deep reactive ion etching (DRIE) to produce a silicon mold 66 with the desired pattern defined by the photolithographic patterning. FIG. 10 is a scanning electron microscope image of a DRIE etched honeycomb patterned silicon mold 66 used to make the non-adherent cell support 22 of FIGS. 7 and 7A. It should be understood that many types of patterns could be used. For example, the vertically extending support members 48 could be plateau-shaped or columnar and have a polygonal or curvilinear cross-sectional shape. In this embodiment, uncured PDMS is poured onto the silicon mold 66, cured, and released from the mold as shown in steps (5) and (6) of FIG. 9 to form the finished non-adherent cell support 22. The non-adherent cell support 22 may be joined to one or more microfluidic chambers, which can be performed using an oxygen plasma treatment. More particularly, the microfluidic chamber sidewalls 26 as shown in FIG. 2 can be subjected to oxygen plasma at 80 Watts for 60 seconds and placed into contact with the non-adherent cell support layer, thereby fusing the two components and forming a completely sealed microfluidic device.

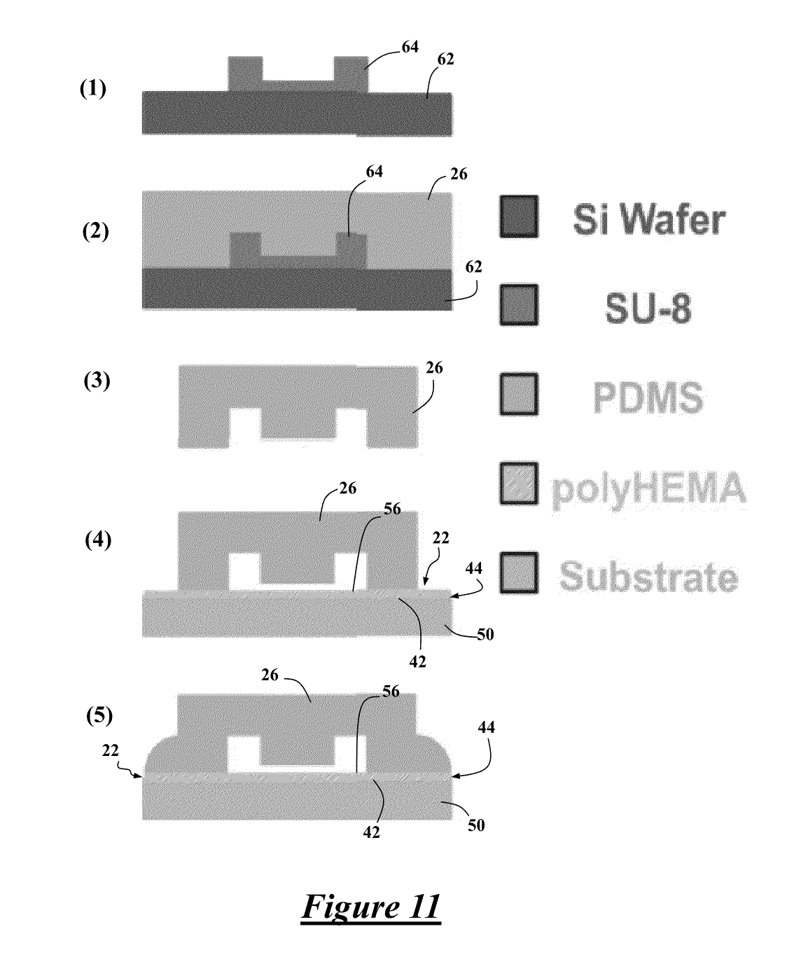

Another method of making a non-adherent cell support 22 is shown in FIG. 11, which depicts an alternate embodiment of a soft lithography method of forming the non-adherent cell support 22 with a biocompatible material. In this particular embodiment, the surface is modified using polyHEMA as the biocompatible material. As shown in steps (1)-(3) of FIG. 11, a silicon wafer 62 is coated with SU-8 photoresist 64 in order to form the microfluidic sidewalls 26 made from PDMS. In step (4), a polyHEMA cell support 22, which can inhibit cell adhesion with or without patterning, is formed on a secondary substrate 50 by slow evaporation. In one particular embodiment, 60 mg/mL of polyHEMA in 95% ethanol is used. In the final step, the PDMS sidewalls 26 and non-adherent cell support 22 are treated by oxygen plasma at 300 Watts for 60 seconds and then bonded together. As an additional step, uncured PDMS may be used as a glue to further fasten the device because polyHEMA swells when exposed to water. This swelling due to the absorption of water may degrade the bonding strength, and thus the PDMS glue (cured at 65 degrees overnight) may minimize this residue stress.

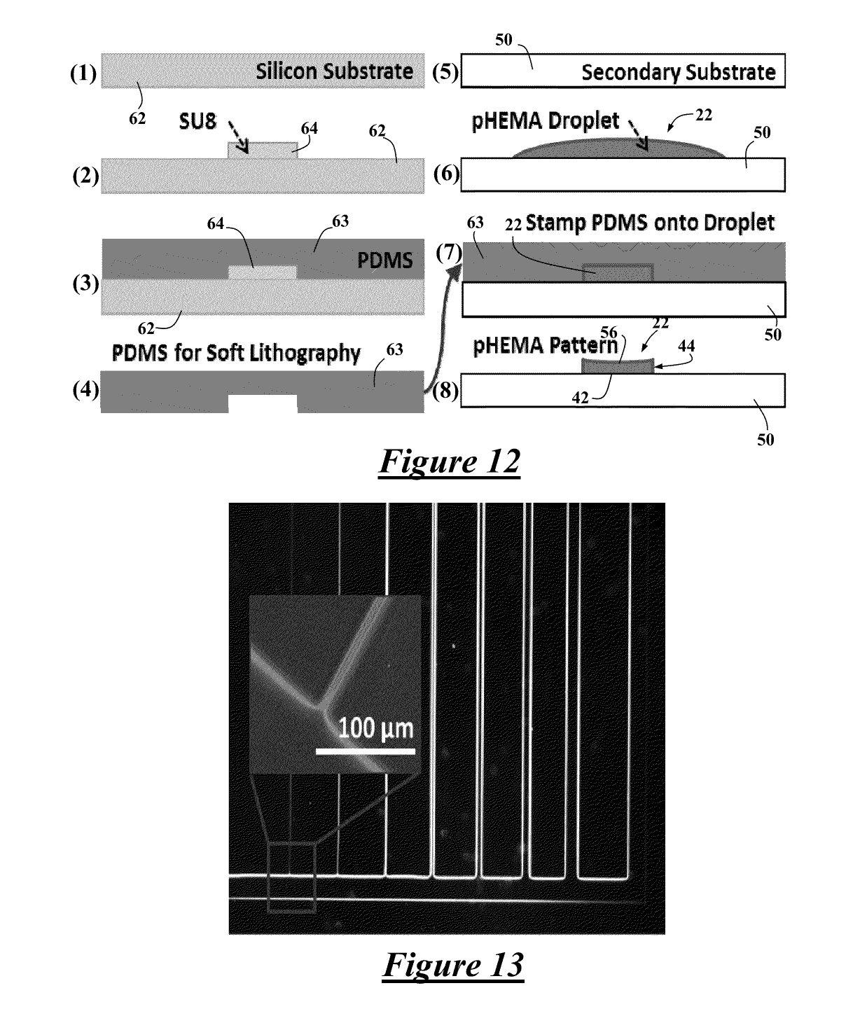

In FIG. 12, yet another method of making a non-adherent cell support 22 is shown, which depicts an alternate embodiment of a soft lithography method of forming the non-adherent cell support 22 with a biocompatible material. Similar to the embodiment described with relation to FIG. 11, the surface is modified using polyHEMA as the biocompatible material. As shown in steps (1)-(4) of FIG. 12, a silicon wafer 62 is coated with SU-8 photoresist 64 in order to form a PDMS stamp 63. In step (1), the silicon wafer 62 is Piranha cleaned. In step (2), the silicon wafer 62 is spun with SU-8 negative photoresist 64 before being patterned by UV-exposure and development. In step (3), PDMS, which may include a curing agent, is poured over the mold, cured, and demolded to create the PDMS stamp 63 in step (4) which is used as the lithographic stamp. In one embodiment there is a 10 to 1 ratio of PDMS to curing agent. In step (5), a secondary substrate 50 is provided, such as a glass plate. In steps (6)-(8), a polyHEMA cell support 22, which can inhibit cell adhesion is formed on the secondary substrate 50. As in the embodiment illustrated in FIG. 11, 60 mg/mL of polyHEMA in 95% ethanol may be used. In step (6), the polyHEMA cell support 22 is deposited on the secondary substrate 50. In one embodiment, 100 μL of the polyHEMA solution is pipetted onto the secondary substrate. The non-adherent cell support 22 is then stamped with the PDMS stamp 63 thereby forming in step (8), a non-adherent cell support 22 with a raised support structure 44 extending upwardly the lower body 42 and forming cell support surface 56. There are three factors that may affect the features of the raised support structure 44 of the pattern: stamp channel height, stamping temperature, and stamping duration. These factors may be manipulated depending on the desired non-adherent cell support, as will be apparent to one having ordinary skill in the art. As shown in FIG. 13, polyHEMA pattern sizes ranging from 2 μm to 500 μm have been demonstrated. This particular embodiment allows for spatial localization of the polyHEMA surface with more precise control of thickness in various profiles.

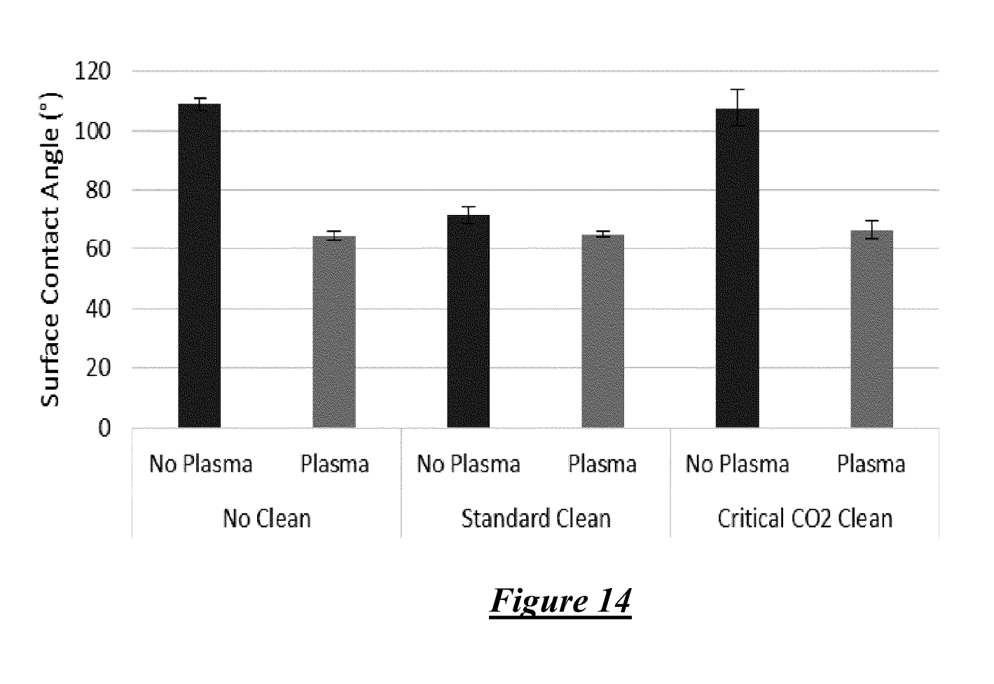

As an optional step to any of the methodologies described above, the non-adherent cell support may be cleaned prior to culturing. This step may be particularly desirable with PDMS cell supports, as PDMS surfaces exhibit mild cell toxicity in long-term cultures. Cleaning the surface prior to culture can remove residual uncured PDMS or silane, thereby causing a significant reduction in this toxicity. FIG. 14 shows the effect of different cleaning procedures on surface contact angle. As will be described in more detail below, a higher surface contact angle is more conducive to non-adherent culturing. Standard cleaning procedures, including cleaning with ethanol or polysorbate surfactants, for example, submerges the PDMS, thermally ages the surface, takes nearly a week to complete, and results in a reduction in contact angle as shown in FIG. 14. Alternatively, the PDMS surface may be subjected to a brief treatment with supercritical carbon dioxide. Supercritical carbon dioxide has low toxicity and a minor environmental impact. Moreover, surfaces cleaned with supercritical carbon dioxide show similar viability to those treated with liquid solvents without a reduction in contact angle, as depicted in FIG. 14. As also shown in FIG. 14, a post-cleaning plasma treatment may be performed. The post-cleaning plasma treatment can reinforce the bonding and may reduce the contact angle to comparable levels, but this effect may fade in a matter of hours after bonding is complete.

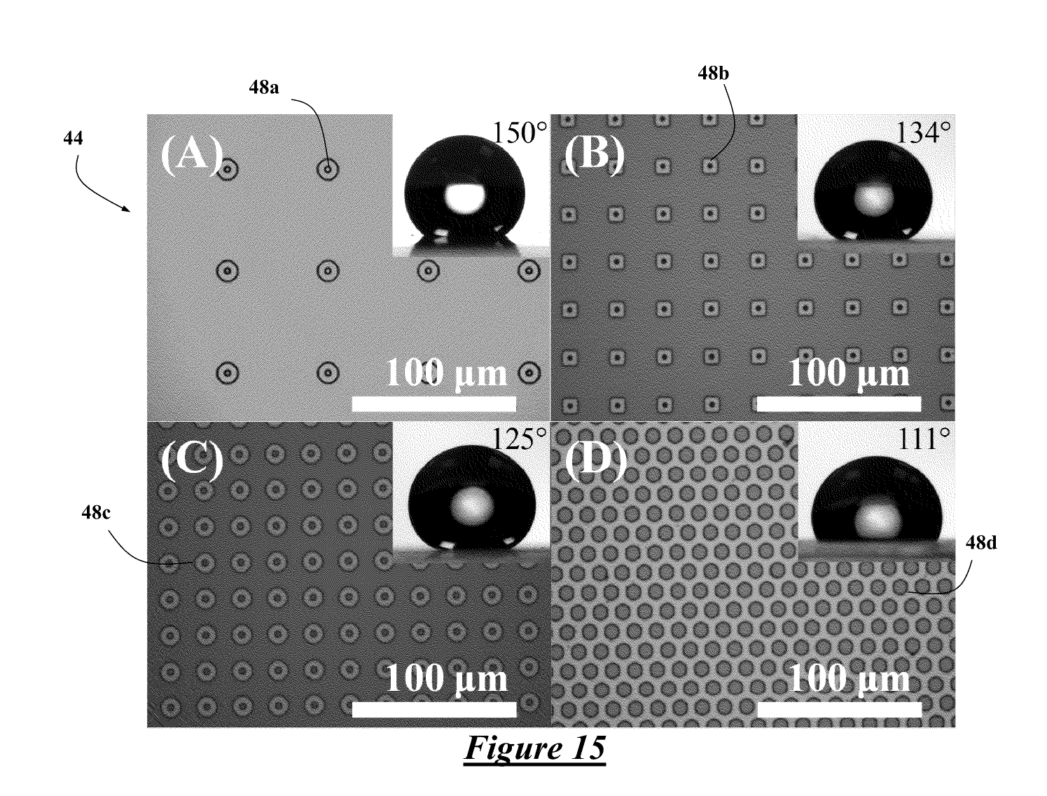

With reference to FIG. 7 and FIG. 7A, the vertically extending support member(s) 48 can comprise a number of different geometries such as the interconnected vertically extending walls. Furthermore, the vertically extending columnar support member or members 48 can have at least one cross-sectional dimension that is between 5.5 and 10 microns. As depicted in FIG. 7, it is possible to have a single, interconnected set of walls forming non-connected voids as indicated by the hexagonal wells. It is also possible to have individual pillars of various shapes and sizes as depicted in FIG. 15. The cross-sectional dimension will vary depending on the desired pattern and shape of the vertically extending columnar support member(s) 48 of the non-adherent cell support 22. Over 15 separate geometries were fabricated with varying pitch, feature size, and shape, some examples of which are shown in FIG. 15. The support structure 44 comprises a patterned array of the one or more support members 48. FIG. 15 shows a few examples of possible shapes that can be used to form the support structure 44, such as a hollow pillar support member 48a, a rectangular shaped support member 48b, and the honeycomb pattern support member 48d as previously discussed. The array of one or more support members 48 can be comprised of a plurality of individual support members 48 laterally spaced from each other forming a connected interstitial space around and between the individual support members 48. The support structure 44 can have a height above the upper surface 46 of the lower body 42 between 10 and 15 microns.

As depicted in FIG. 15 each separate geometry of the designed support pattern 44 showed a different hydrophobicity. The hydrophobicity was tested by measuring the contact angle of 10 μL droplets of water on the surface, and the resulting contact angles are shown in FIG. 15. The contact angle of the surfaces varied from 111° to 150° depending on their geometries. The hollow pillar shaped support members 48a with a larger interstitial space resulted in the highest contact angle of 150°. The rectangular shaped support members 48b resulted in a contact angle of 134°. The hollow pillar shaped support members 48c resulted in a contact angle of 125°, and the honeycomb shaped vertically support member 48d resulted in the lowest contact angle of 111°. It was observed that contact angles increased with increasing pitch and decreasing pillar size. Superhydrophobicity (a contact angle greater than 150°) was achieved using both 5 and 10 μm pillars with a pitch of 50 μm.

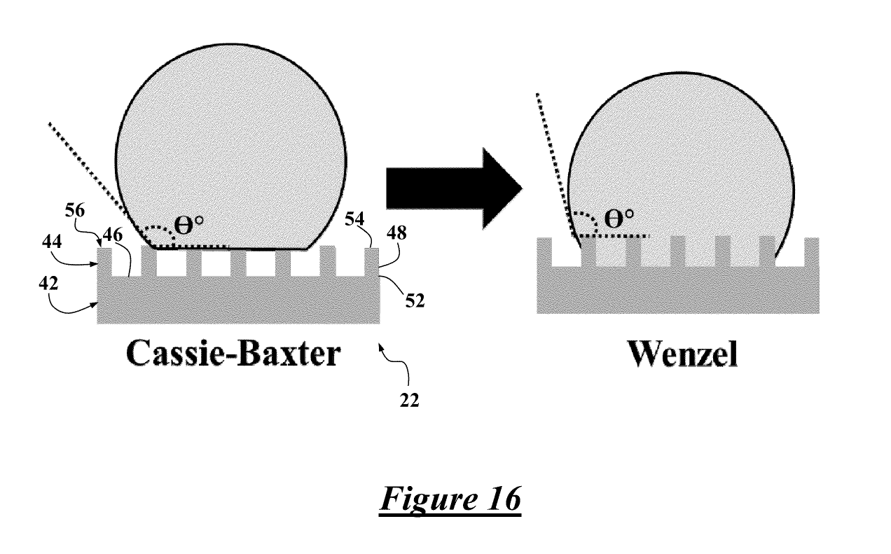

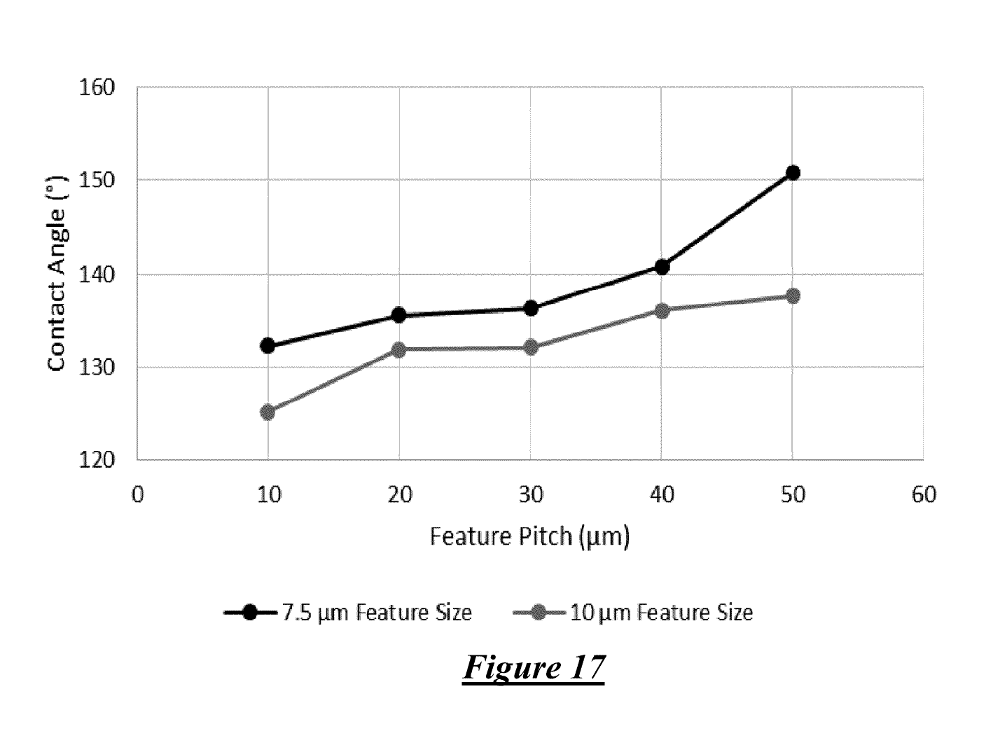

The pattern pitch of the patterned array of one or more support members 48 should vary between 10 and 50 microns. A pitch that is too high on an unconnected surface could be susceptible to Cassie-Baxter to Wenzel state transitions, as depicted in FIG. 16. The non-adherent cell support 22 should keep the cell or fluid in the Cassie-Baxter state. Air trapped underneath the fluid minimizes the contact area of the cells. To maintain the Cassie-Baxter state, fluid contact line forces must overcome body forces of unsupported droplet fluid weight, and the support members 48 must be tall enough to prevent the liquid that bridges support members 48 from touching the base of the support member 48 as shown in the Wenzel state in FIG. 16. The ratio of the area of trapped air compared to the area of the contacting surface is relevant in determining hydrophobicity. As shown in FIG. 17, increasing pitch between support members reduces the contact surface area and therefore increases the resulting contact angle. Decreasing the size of the support members similarly causes an increase in contact angle. These relationships should remain true so long as the surface remains in the Cassie-Baxter state. Increasing contact angle, which indicates higher hydrophobicity, should better repel cells and biofouling factors. However, reducing the contact area may also make the surface more vulnerable to Cassie-Baxter to Wenzel state transitions, resulting in a lack of ability to prevent cell attachment.

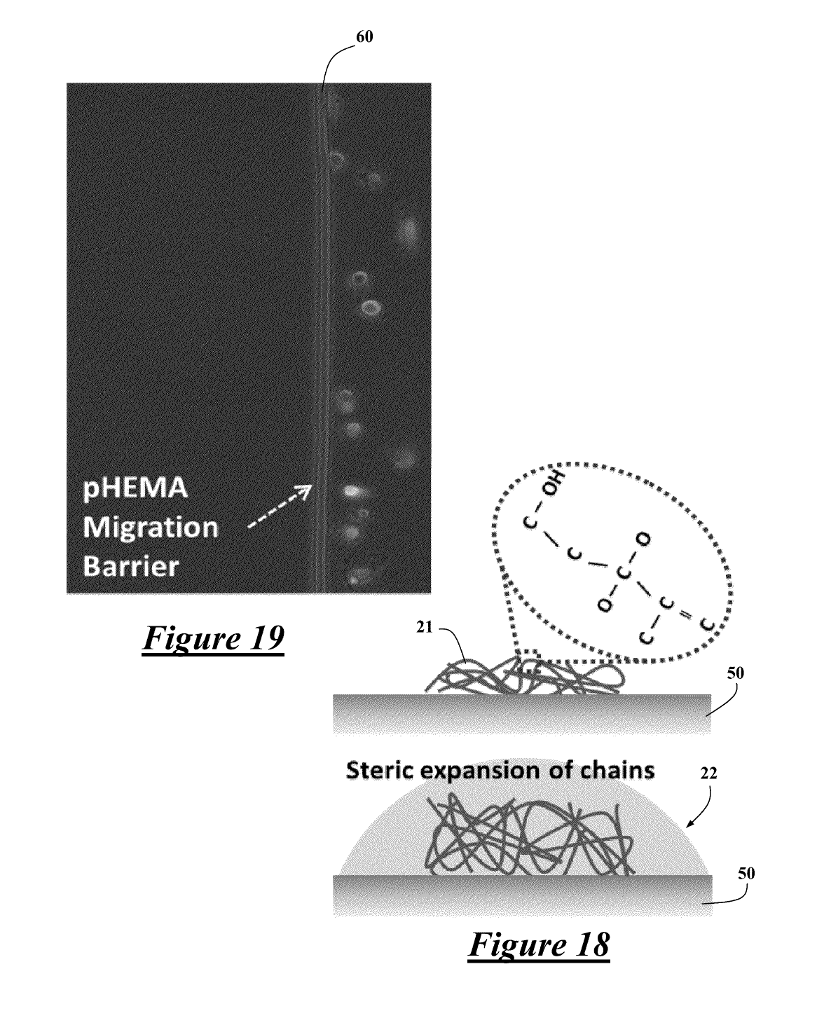

FIG. 18 shows an embodiment of the present invention using polyHEMA as the biocompatible material. A non-aqueous polyHEMA support 21 is shown on substrate 50. When polyHema is in non-aqueous environments, a non-polar methyl group is turned outward making it hard and compact. However, an aqueous polyHEMA support 22 absorbs water and the hydroxyethyl side turns outward, facilitating flexibility and swelling of the polymer chains. The expanded chains sterically block cell adhesion by preventing the cell from interacting from the substrate 50. It is also possible to construct small polyHEMA walls as migration blocks in culture. As shown in FIG. 19, MDA-MB-231 breast cancer cells seeded onto the right side of polyHEMA barrier 60 were unable to migrate across. In one particular embodiment, a polyHEMA barrier that is 3 μm tall was incorporated into single cell clonal culture microfluidic devices to prevent well-to-well migration. The polyHEMA barriers were able to constrain cell growth without significantly disrupting gravity flows in the channels.

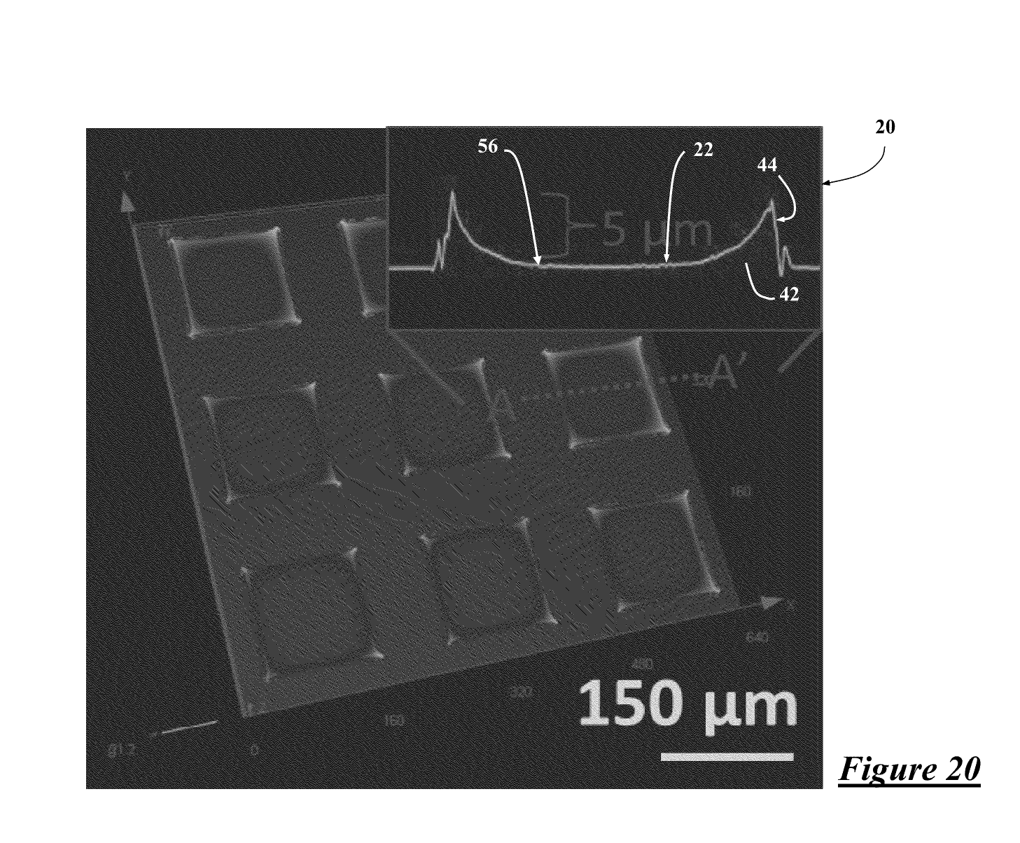

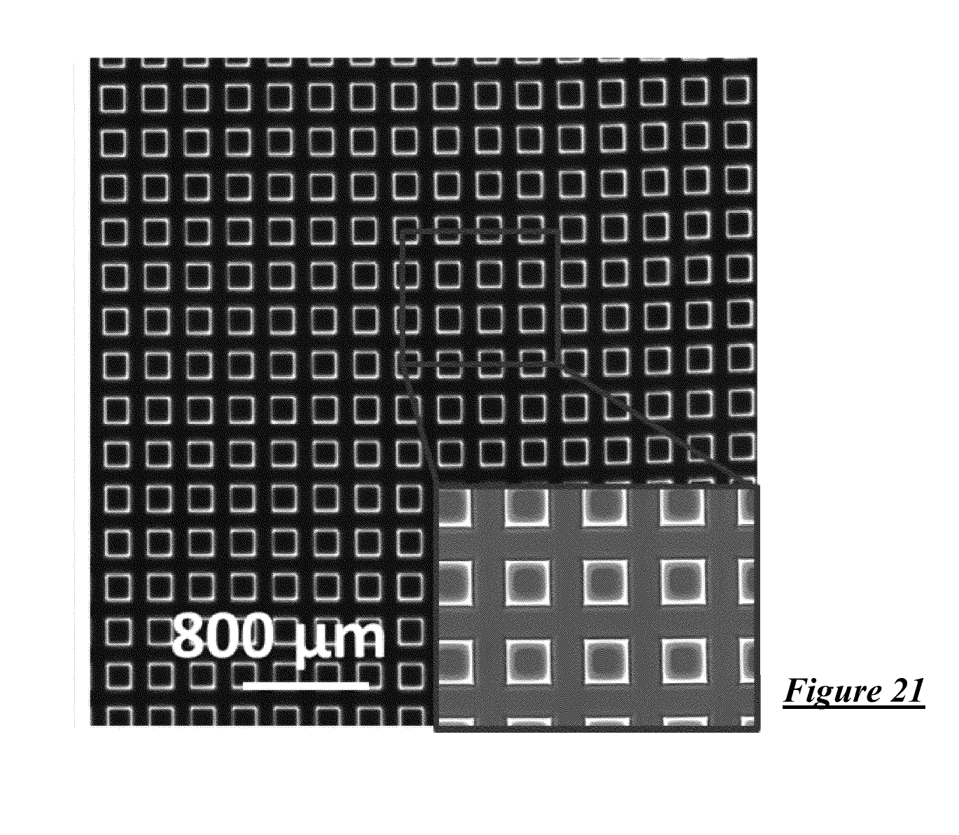

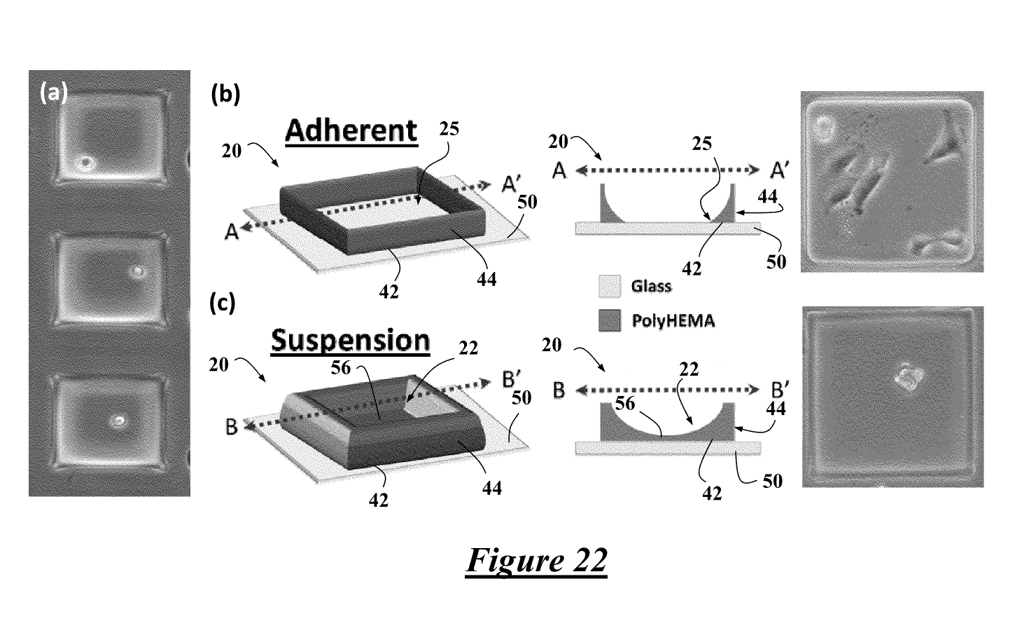

A non-aqueous polyHEMA support may serve as a reusable master for further PDMS lithography. This approach may have several advantages. First, the chemical properties of non-aqueous polyHEMA facilitate de-molding of small PDMS features without any silanization. This may be beneficial for culture applications with sensitive cells (such as single cell culture or primary cells directly from patients), where the residual silane decreases viability. Additionally, by controlling stamping temperature, it is possible to create concave features in the deposited polyHEMA, as shown in FIGS. 20-22. These types of features may be useful in microfluidic valves, pumps, and even microlenses, and are often difficult to create with standard microfabrication technologies. With particular reference to the subset in FIG. 20, a polyHEMA microwell 20 may consist of non-adherent cell support 22 consisting of cell support member 44 with lower body 42 and cell support surface 56. These concave microwells depicted in FIGS. 20-22 may be created by depositing polyHEMA at elevated temperatures. In one particular embodiment, the polyHEMA is deposited at temperatures in excess of 50° C. As shown in FIG. 22, the thickness progressively increases from the center of the microwell 20 toward the edges due to wall-fluid interactions. This effect may be enhanced at increased temperatures, such as temperatures in excess of 80° C., resulting in a complete depletion of polyHEMA in an adherent microwell cell support 25. SUM159 breast cancer cells were loaded into the microwells 20. Those grown with an adherent microwell cell support 25 grew into adherent colonies as shown on the right. Those grown on a non-adherent cell support 22 grew into spheroids. In some cases, the settling procedure is probabilistic, so it may be beneficial to use microwell arrays with higher numbers of microwells so as to increase the probability that single cells will be captured in the individual microwells. It is possible to remove residual cells through gentle washing. The high-throughput arrays as shown in FIGS. 20 and 21 may be useful in a variety of cell culture assays including those used for single cell phenotyping, clonal analysis, and spheroid drug assays, for example.

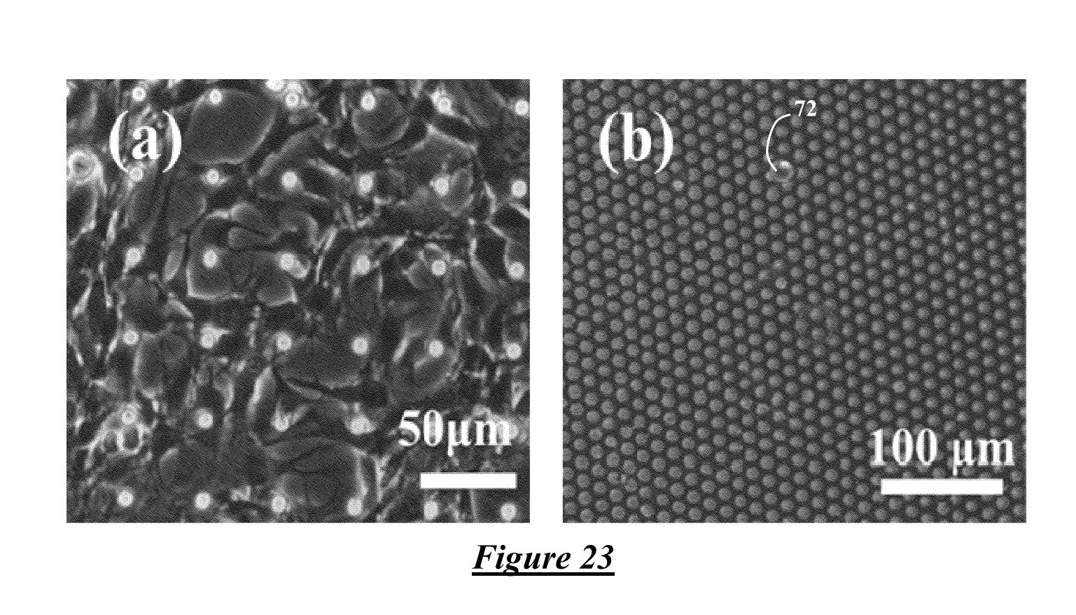

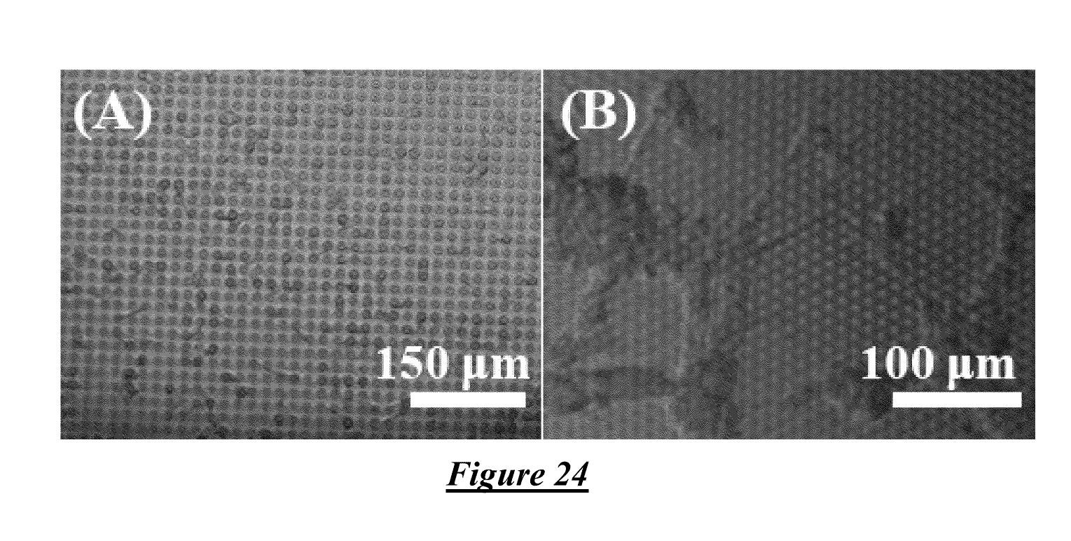

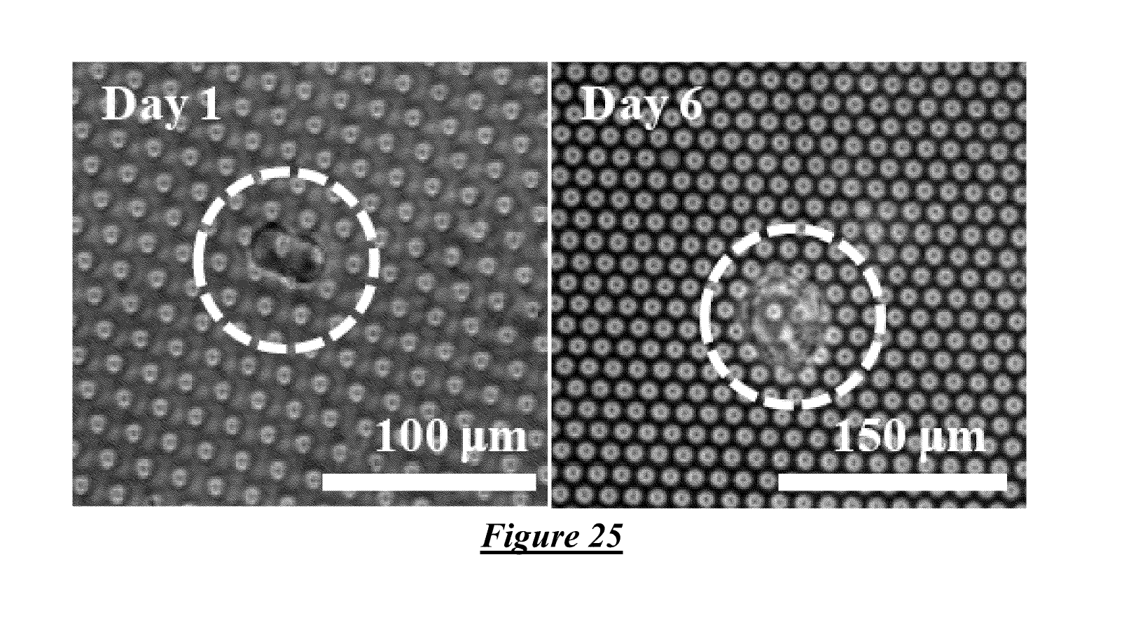

FIGS. 23-26 depict cultures of three different types of cells: C2C12 myoblasts, 10T1/2 fibroblasts, and SUM159 breast cancer cell lines. These cell lines were chosen as they have particular surface requirements: C2C12 undergoes adherent culture only, 10T1/2 prefers adhering to a surface but also grows as aggregations, and the CSC subset of SUM159 cancer cells are capable of suspension sphere culture. Prior to loading the cells into the microfluidic chambers, the three different types of cells were cultured in Dulbecco's Modified Eagle Medium with 10% fetal bovine serum. For loading the cells into the microfluidic chambers, trypsin with 0.05% ethylenediaminetetraacetic acid was used to suspend the cells. After loading, the cells were cultured for spheroid formation on the non-adherent cell support surfaces by switching the culture media to mammary epithelial cell basal medium with additional supplements including B27, insulin, lipid concentrate, hydrocortisone, cholesterol, epidermal growth factor, and basic fibroblast growth factor. FIGS. 23-24 are arranged such that a surface that allows for attachment (i.e., transitions from the Cassie-Baxter to Wenzel state) is disposed on the left, and is compared to a non-adherent honeycomb shaped support on the right. As shown in FIG. 23, because the C2C12 undergoes adherent culture only, there was good attachment on the left and death, shown as cellular debris 72 and a lack of adherent cells, on the right. With reference to FIG. 24, because the 10T1/2 prefers adhering to a surface but also grows as aggregations, there was good attachment on the left and aggregation formation on the right. FIG. 25 shows successful growth of SUM159 spheres over the course of 6 days cultured on the right. Only the CSC subset of SUM159 were capable of forming spheres from single cells and spheres were formed in a ratio (approximately 42% of the total loaded cells) which is comparable to the current inefficient and expensive FACS single cell culture method in specialized non-adherent 96-well plates.

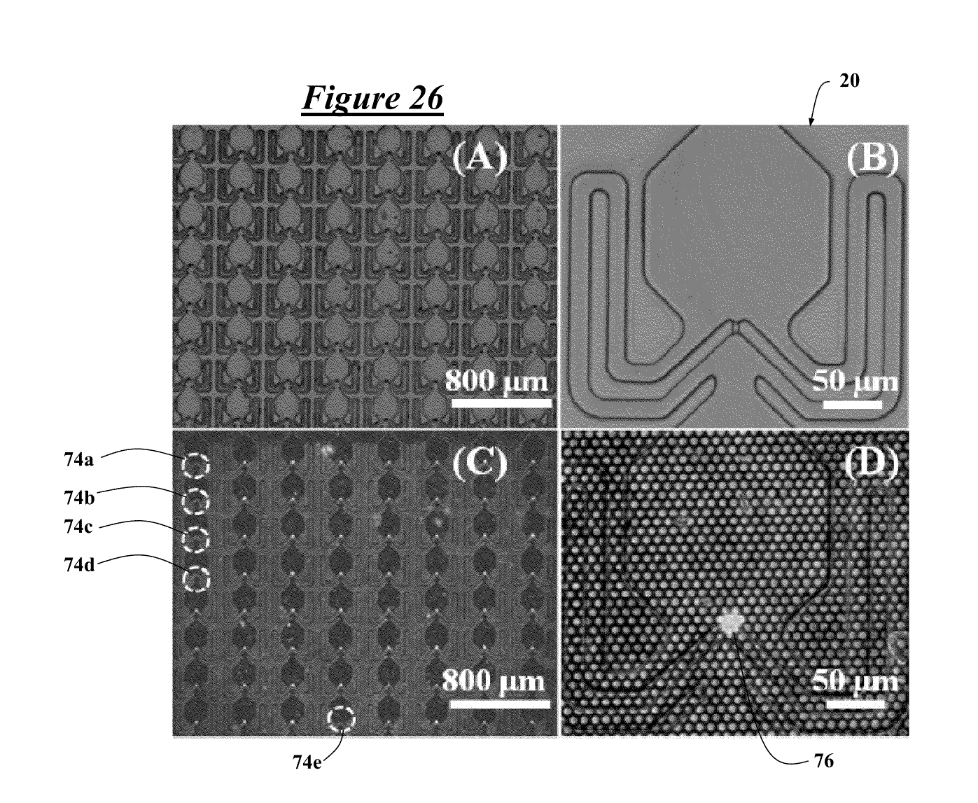

FIG. 26 depicts captured SUM159 cells in a microchamber. More particularly, Section (A) provides an overview of a high-throughput single-cell capture device comprising 64 microfluidic chambers 20. Section (B) is a magnified view of the single microfluidic chamber 20 for hydrodynamic single cell capture. Section (C) shows SUM159 cells captured in a honeycomb-surface single cell capture device. The capture rate was approximately 92% (59 of 64 chambers). White circles 74 indicate the few chambers where no cell was captured. Section (D) shows a honeycomb surface integrated with single cell capture platform for high throughput CSC culture. Fluorescence area 76 indicates viable SUM159 that was grown in the device for 3 days.

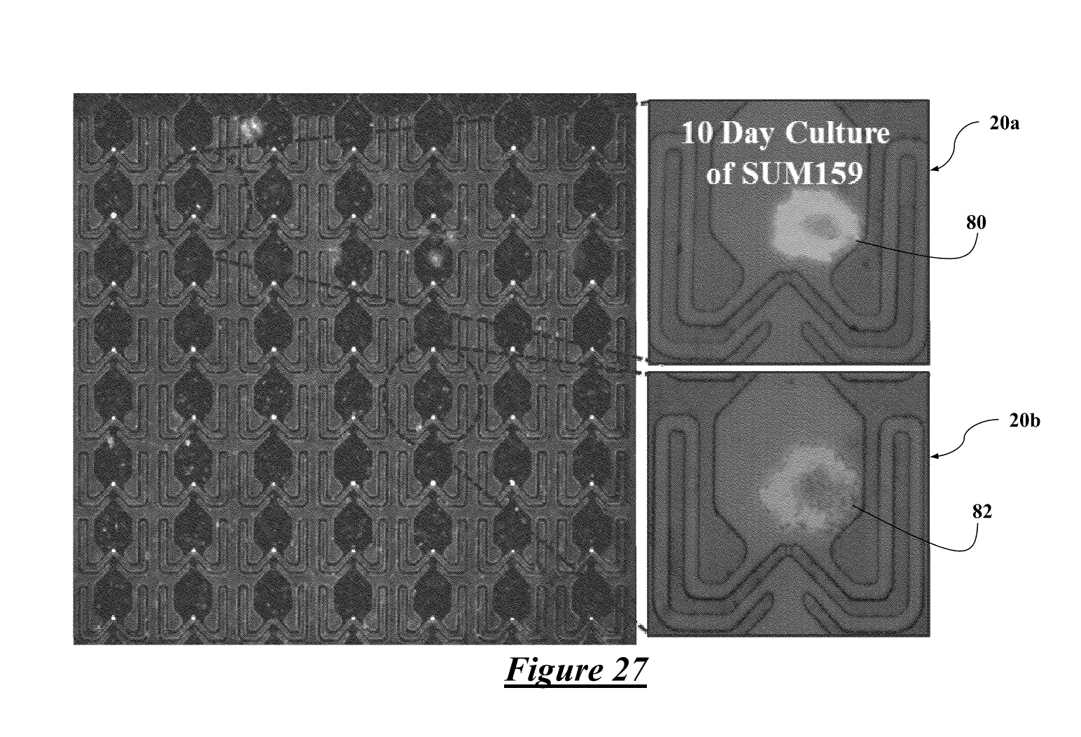

FIG. 27 shows a single cell derived spheroid formation in a plurality of microfluidic chambers 20. SUM159 cells were captured at a rate exceeding 90%, and they were grown for 10 days. Forty-two percent of all chambers 20 formed spheres, an efficiency comparable to traditional methods. Enlarged views of two chambers 20a and 20b show SUM159 sphere growth 80 and 82, respectively. In one particular experiment, 54.69% of wells formed single-cell derived spheres, compared with 55.73% using the time consuming, expensive, conventional methodology. In another comparable experiment, the microfluidic chambers with non-adherent cell support allowed observation of MDA-MB-231 single-cell derived sphere formation. Although only 1.28% of wells formed single-cell derived cultures, no wells formed MDA-MB-231 single-cell derived spheres using conventional methodology.

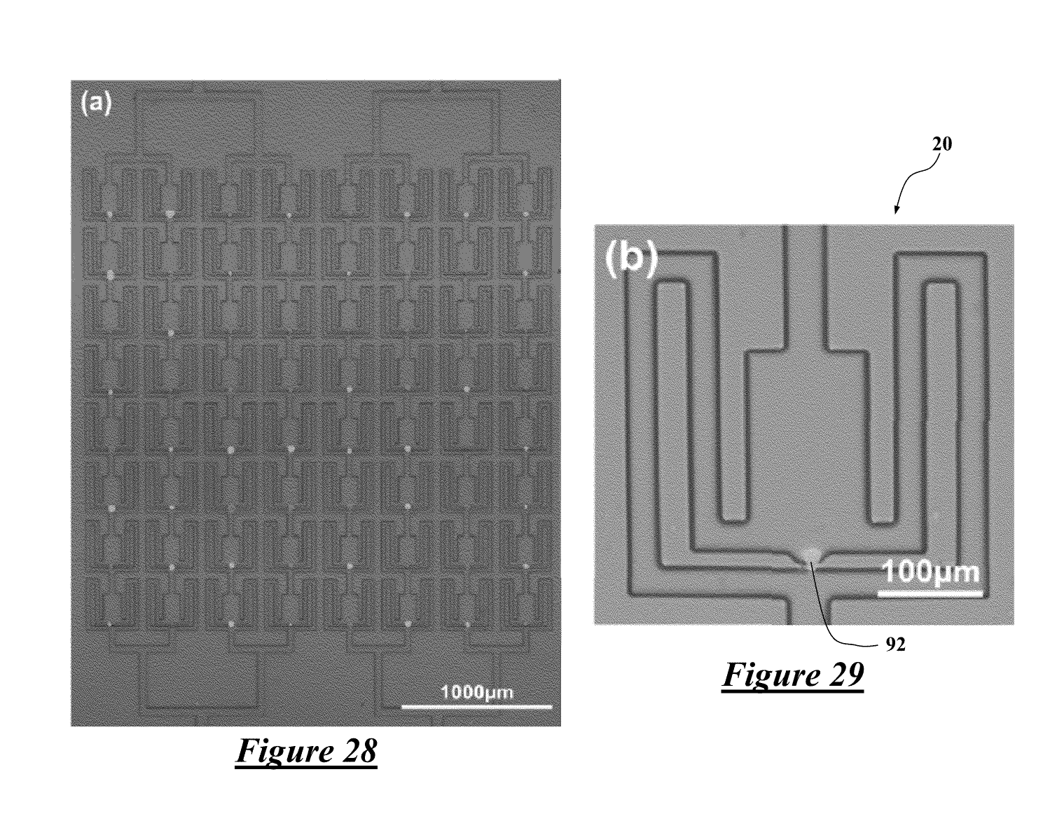

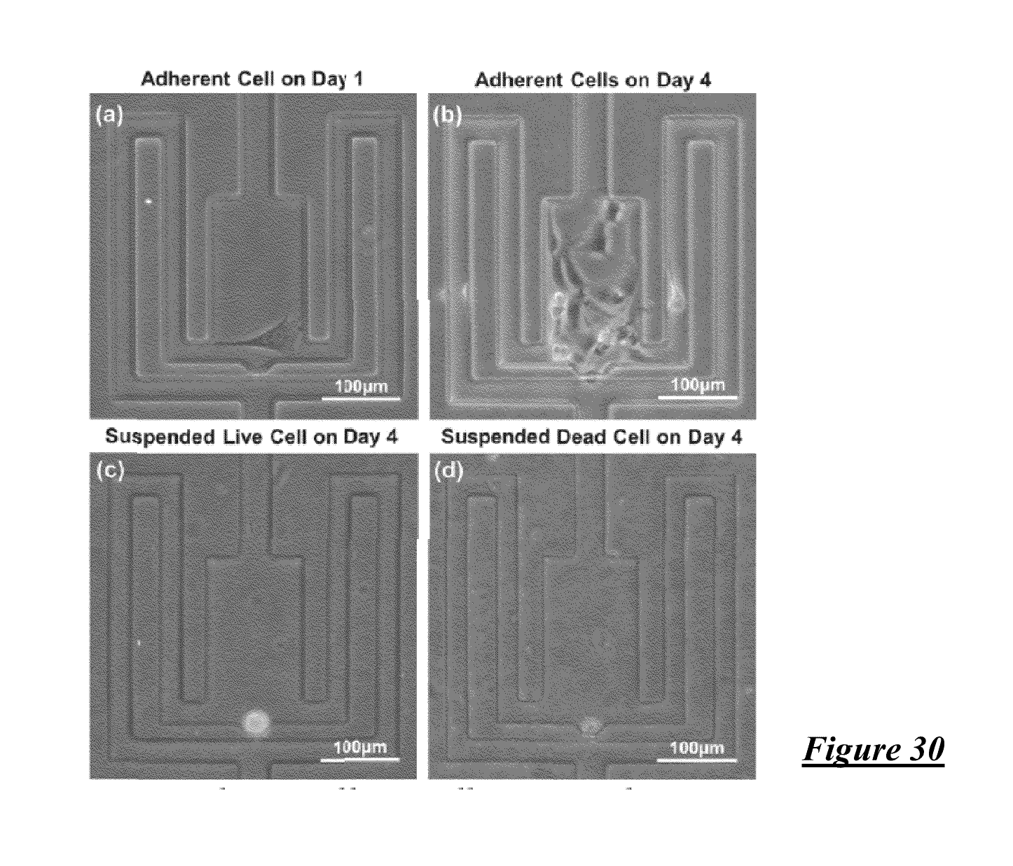

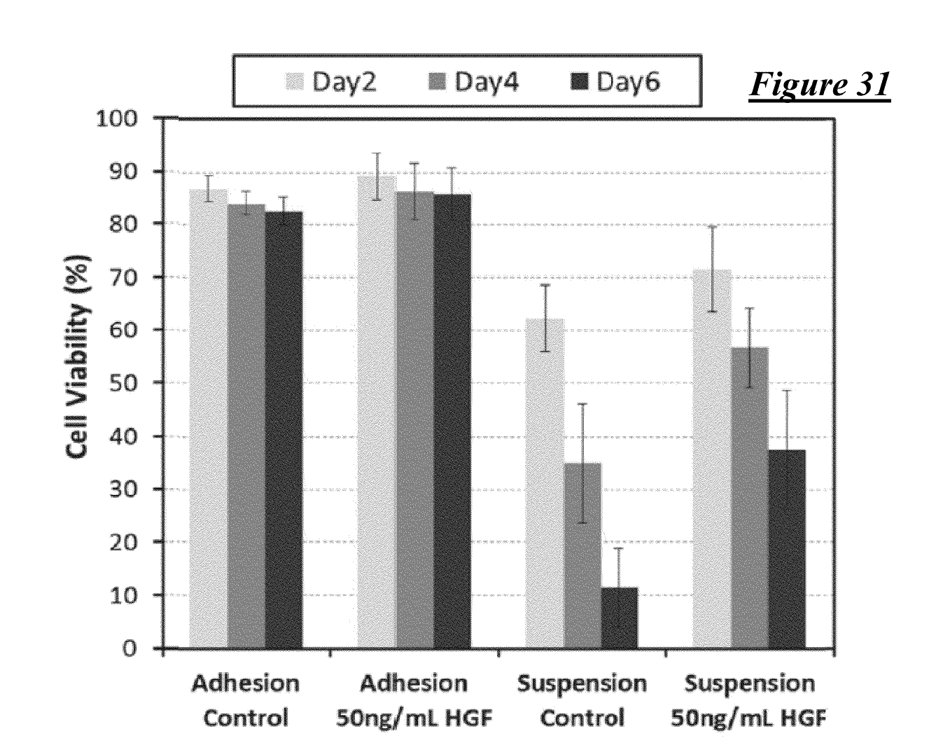

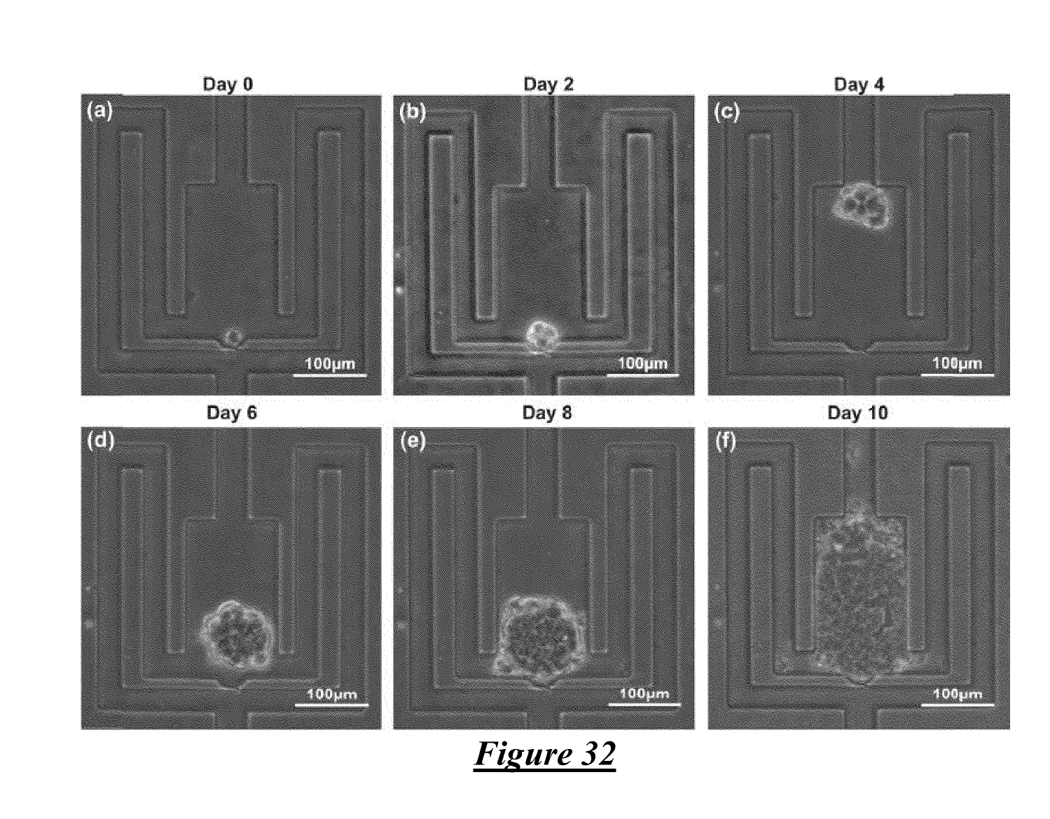

FIG. 28 shows a microchamber array where polyHEMA is used as the non-adherent cell support. In this particular embodiment, there was a single-cell capture rate over 90%. A single microchamber 20 from the microchamber array of FIG. 28 is shown in FIG. 29, where a single skov3 ovarian cancer cell 92 is captured. An anoikis assay of skov3 ovarian cancer cells, which are known to not grow in suspension, is shown in FIG. 30, which compares suspension culture and adherent culture. The cells were treated with hepatocyte growth factor (HGF) which is believed to enhance cell survival in suspension culture. The attached skov3 cells proliferated during the four day culture, while the suspended cells underwent apoptosis. FIG. 31 summarizes the anoikis experiments, and the results confirm that an enhanced survival rate may be observed when cells are exposed to 50 ng/mL HGF. FIG. 32 shows an experiment with sphere formation from single SUM159 cells using microchambers that include polyHEMA as the non-adherent cell support. In this particular trial, 72% of SUM159 cells in the microchambers formed spheres after ten days.

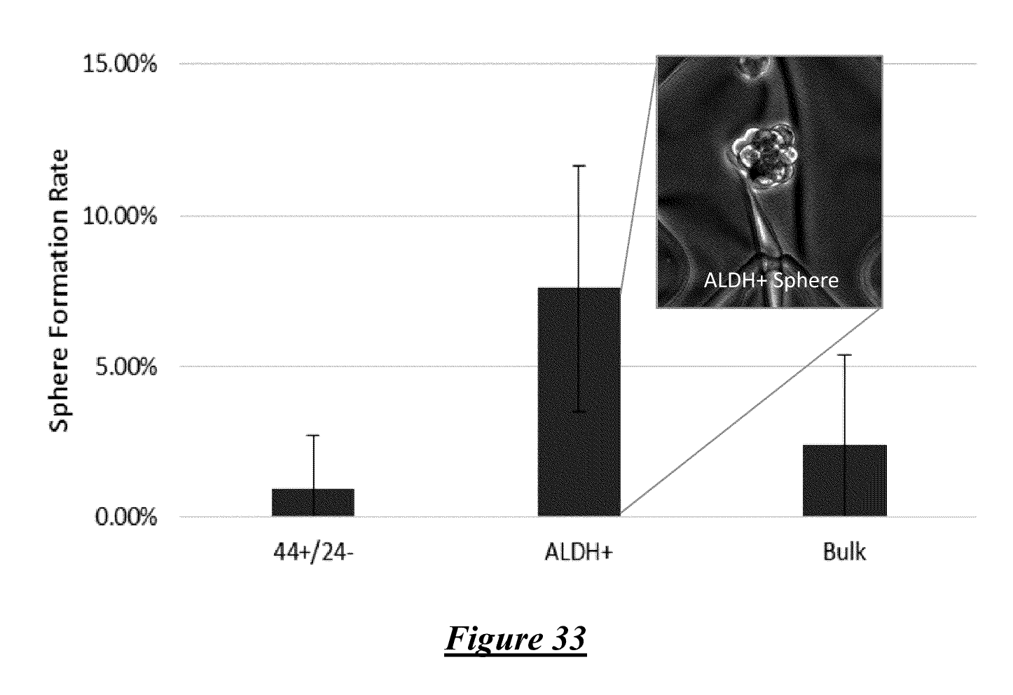

The non-adherent cell supports described herein may also allow for the assessment of sub-population behavior within a single cell type. This capability is beneficial as often there are multiple, independent markers that all may be associated with stem cell-like characteristics. As shown in FIG. 33, the sphere formation rates of the sub-populations of T47D breast cancer cells were characterized. T47D breast cancer cells have CD44+/CD24− and ALDH+ independent, non-overlapping populations that both exhibit stem-like characteristics. These sub-populations of T47D cells were sorted and sphere forming potential was assessed to evaluate possible tumorigenic ability. This can facilitate better evaluation of which sub-populations may contribute more to metastasis and/or tumor growth. An experiment was conducted by FACS sorting the T47D populations and loading them into microwells with non-adherent cell support. As shown in FIG. 33, ALDH+ T47D cells may be a greater contributor to sphere formation compared to CD44+/CD24− or bulk (i.e., non-sorted) cells.

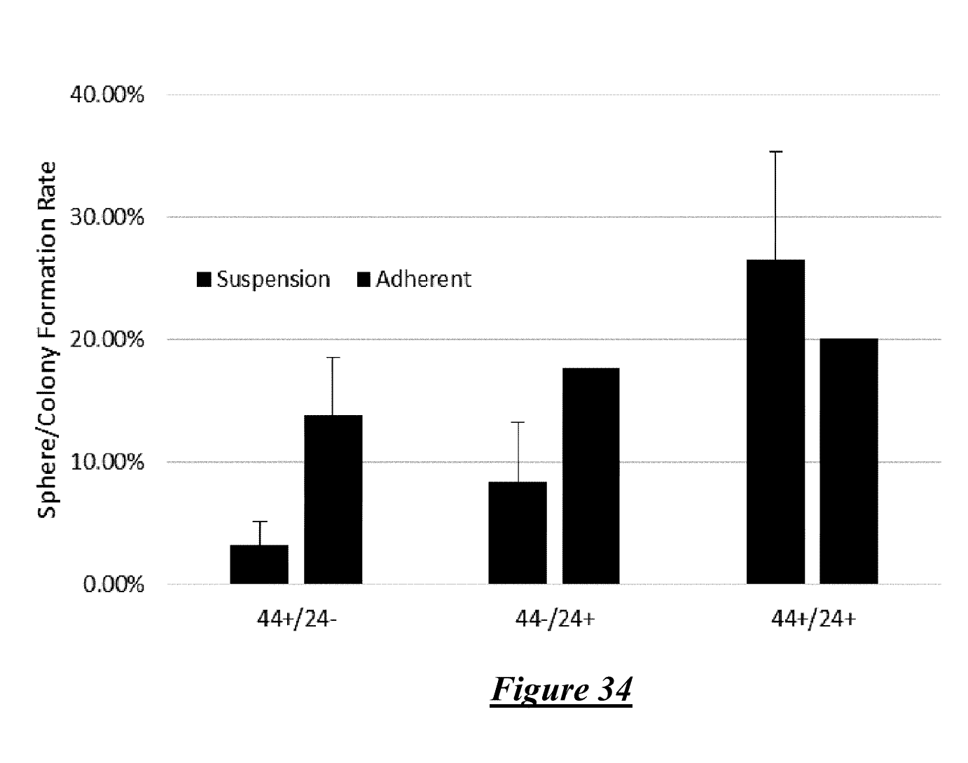

The non-adherent cell supports' ability to allow for precise spatial localization provides another benefit over conventional culturing methods. For example, a subset of microwells can be patterned for suspension culture while others can be utilized for adherent culture. This facilitates easier side-by-side comparison of differences in suspension and adherent growth potential. As shown in FIG. 34, and described above, CD44+/CD24− cells may have a large increase in growth potential when allowed to biofoul the surface and attach. However, when cultured on a semi-adherent environment that allows them to secrete extracellular matrix (ECM) and attach, a significant increase in growth and colony number is possible. Comparatively, CD44+/CD24+ progenitor cells may have no significant difference in growth potential between suspension and adherent conditions. Because CD44+/CD24+ cells are further differentiated than CD44+/CD24− stem-like cells, they can more easily transition to facilitate survival in both conditions.

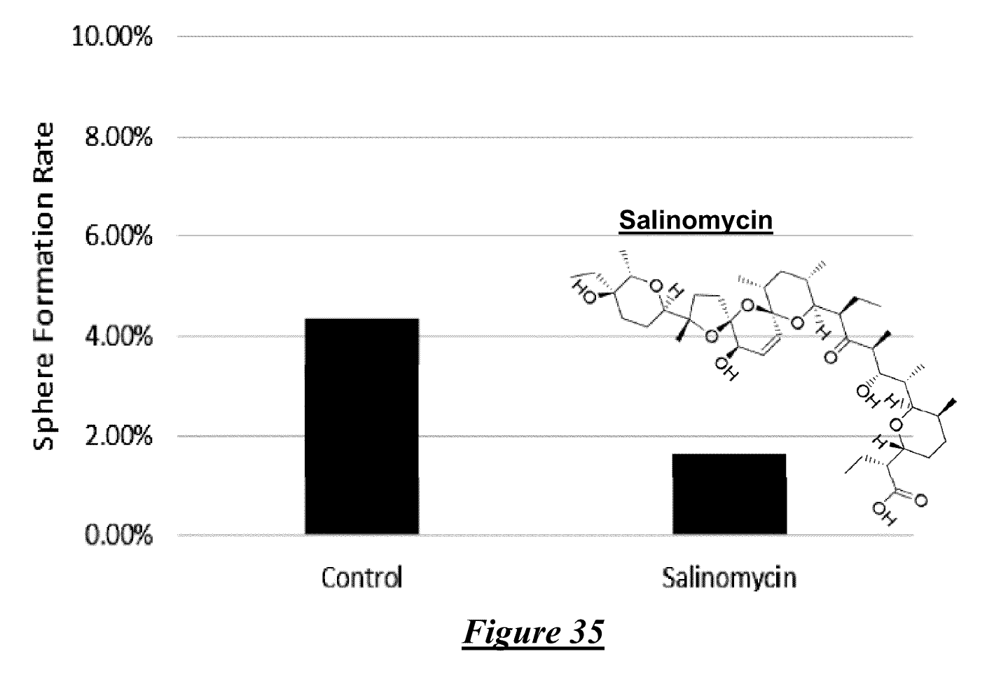

Furthermore, microassays using the non-adherent cell support for single-cell derived sphere formation may be used as a readout indicator for CSC-targeted drug screening. In one particular experiment, T47D cells were treated with salinomycin and normal culture media for 1 day prior to sphere formation. The resulting rates were recorded and a decrease in sphere formation in the salinomycin treated cells was observed, as depicted in FIG. 35.

It is to be understood that the foregoing description is of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A non-adherent cell support for use in cell assays, comprising:
a hydrophobic substrate having a lower body and a raised support structure extending upwardly from an upper surface of the body, the support structure comprising one or more vertically extending support members that extend from a proximal portion at the upper surface of the body to a distal end spaced from the upper surface of the body, the distal end of the one or more support members forming an interrupted support surface for hydrophobic support of cells on the support structure.

2. The non-adherent cell support of claim 1, wherein the lower body comprises a biocompatible material and the support structure comprises a continuous extension of the lower body biocompatible material that extends upwardly from the upper surface of the lower body to the support surface at the distal end of the one or more support members.

3. The non-adherent cell support of claim 2, wherein the biocompatible material is poly-2-hydroxyethyl methacrylate, polydimethylsiloxane, polymethyl methacrylate, polystyrene, or a polyethylene glycol diacrylate-based hydrogel.

4. The non-adherent cell support of claim 2, wherein the biocompatible material is poly-2-hydroxyethyl methacrylate and the one or more support members has a plateau shape.

5. The non-adherent cell support of claim 1, wherein the support structure comprises a patterned array of the one or more support members.

6. The non-adherent cell support of claim 5, wherein the array of one or more support members comprises a plurality of individual support members laterally spaced from each other forming a connected interstitial space around and between the individual support members.

7. The non-adherent cell support of claim 6, wherein the individual vertically extending support members are columnar and have a polygonal or curvilinear cross-sectional shape.

8. The non-adherent cell support of claim 7, wherein the columnar support members have at least one cross-sectional dimension that is between 5.5 and 10 microns.

9. The non-adherent cell support of claim 5, wherein the array of one or more support members comprises a pattern of interconnected vertically extending walls forming a plurality of non-connected open voids at least partially defined by the interconnected walls and upper surface of the lower body.

10. The non-adherent cell support of claim 9, wherein the interconnected walls form a honeycomb pattern.

11. The non-adherent cell support of claim 5, wherein the patterned array of one or more support members has a pattern pitch between 10 and 50 microns.

12. The non-adherent cell support of claim 1, wherein the support structure has a height above the upper surface of the lower body that is between 10 and 15 microns.

13. A microfluidic chamber for use in individual cell assays, comprising:
a non-adherent cell support as defined in claim 1;
a chamber upper wall spaced from said non-adherent cell support and at least partially defining an interior region;
a chamber sidewall structure including at least one sidewall extending downwardly from said upper wall toward said non-adherent cell support so as to at least partially define the interior region, said chamber upper wall and chamber sidewall structure together comprising a cell microchamber attached to said non-adherent cell support; and
a front valve and a rear valve, wherein said front valve comprises a first actuator and a first section of said sidewall structure located at a fluid entry point for said microchamber, and wherein said rear valve comprises a second actuator and a second section of said sidewall structure located at a fluid exit point for said microchamber, each of said valves being controlled via its associated actuator to permit said valves to be switched between open, neutral, and closed positions, with the neutral position for each valve permitting fluid flow through the valve while preventing cell transference through the valve, the open position for each valve permitting fluid flow and cell transference through the valve, and the closed position preventing both fluid flow and cell transference through the valve.

14. A macro-scale chamber comprising the non-adherent cell support of claim 1.

15. A non-adherent cell support for use in cell assays, comprising:
a substrate having a lower body and a hydrophobic support structure extending upwardly from an upper surface of the body, the support structure comprising one or more support members that extend from a proximal portion at the upper surface of the body to a distal end spaced from the upper surface of the body, wherein the support structure is formed from poly-2-hydroxyethyl methacrylate.

16. The non-adherent cell support of claim 15, wherein at least one of the one or more support members has a concave cell support surface.

17. The non-adherent cell support of claim 16, wherein the concave cell support surface meets the lower body to form an area capable of adhesive culturing.

18. A microfluidic chamber for use in individual cell assays comprising the non-adherent cell support of claim 15.

19. A macro-scale chamber comprising the non-adherent cell support of claim 15.

20. A method of making a microfluidic device having a non-adherent cell support for use in cell assays, comprising the steps of:
fabricating one or more microfluidic chamber structures and a non-adherent cell support; and
joining one or more microfluidic chambers to the non-adherent cell support.

21. The method of claim 20 wherein the non-adherent cell support is joined to the one or more microfluidic chamber structures using an oxygen plasma treatment.

22. A method of making a microfluidic device having a non-adherent cell support for use in cell assays, comprising the steps of:
providing a silicon wafer;
spin coating and patterning said silicon wafer with photoresist;
deep reactive ion etching the coated silicon wafer to produce a patterned mold;
pouring an uncured biocompatible material onto the patterned mold resulting in an uncured non-adherent cell support;
curing the uncured non-adherent cell support;
releasing the cured non-adherent cell support from the patterned mold; and
joining the non-adherent cell support to one or more microfluidic chambers.

23. The method of claim 22 wherein the biocompatible material is poly-2-hydroxyethyl methacrylate, polydimethylsiloxane, polymethyl methacrylate, polystyrene, or a polyethylene glycol diacrylate-based hydrogel.

24. The method of claim 22 wherein the non-adherent cell support is joined to one or more microfluidic chambers using an oxygen plasma treatment.

25. The method of claim 22, further including the step of cleaning the non-adherent cell support with supercritical carbon dioxide.
